(19) United States
(12) Patent Application Publication
(10) Pub. No.: US 2026/0315081 A1
(43) Pub. Date: Oct. 15, 2026

(54) Intramedullary Targeting Guide Assembly and Related Methods

(71) Applicant: VOOM Medical Devices, Inc., New York, NY (US)

(72) Inventors: Neal Blitz, New York, NY (US); Dan Avni-Baifus, Sherman Oaks, CA (US); Dustin E. Campbell, Nashville, TN (US); Bogdan Grecea, Los Angeles, CA (US)

(21) Appl. No.: 19/578,823

(22) Filed: Mar. 25, 2026

Related U.S. Application Data
(60) Provisional application No. 63/777,609, filed on Mar. 25, 2025.

(51) Int. Cl. A61B 17/17; A61B 17/00; A61B 17/56; A61B 90/00

(52) U.S. Cl. CPC A61B 17/1717; A61B 17/1775; A61B 90/39; A61B 2017/00407; A61B 2017/00477; A61B 17/56; A61B 2017/565; A61B 2090/3966

(57) ABSTRACT

An intramedullary targeting guide assembly configured to guide bone screw placement in orthopedic surgery, including a clamp, a rail member, a drill guide assembly, a rotation assembly, and a vertical shift assembly. The clamp (or “forceps clamp”) is configured to register the intramedullary targeting guide assembly to a bone by clamping to the cortical wall of the bone. More specifically, the clamp includes a first portion (e.g., intramedullary tip jaw) configured to extend at least partially into the intramedullary canal of the bone through an osteotomy opening and a second portion (e.g., shovel tip jaw) configured for positioning extramedullary but subcutaneously such that a portion of the cortical wall of the bone is situated between the first portion and the second portion. The clamp can be actuated such that the first and second portions forcibly engage the cortical wall to secure the clamp to the bone.

### CROSS-REFERENCES TO RELATED APPLICATIONS

The present application claims the benefit of priority from U.S. Provisional Patent Application No. 63/777,609, filed Mar. 25, 2025, and entitled “Intramedullary Targeting Guide Assembly and Related Methods,” the entire contents of which are hereby incorporated by reference into this disclosure as if set forth fully herein.

### FIELD

The present disclosure relates generally to surgical systems, and more specifically to a targeting guide system and method for precise screw placement during minimally invasive bunion surgery.

### BACKGROUND

A bunion typically appears as a bony bump near or at the base of the metatarsophalangeal (MTP) joint of the big toe. As a result, the big toe pushes against the neighboring toe, causing the MTP joint to protrude laterally. In some cases, the MTP joint of the big toe can become dislocated. If a surgical solution is sought, the metatarsal bone of the big toe is severed and realigned into proper position. Once the bone has been realigned, however, a portion of the severed metatarsal bone, also referred to as “prominent redundant bone”, protrudes laterally from the foot. This prominent redundant bone portion is then cut and removed. The cut bones are then secured using any combination of fasteners, including for example screws, pins, or plates. The final result is a bunion bone that is shifted into proper alignment with the shaved bone on the side of the foot, so the inside of the foot is straight and narrow, without any bony protrusions.

Typical minimally invasive bunion surgery is performed through two tiny incisions using specialized instruments. One incision (e.g., “distal incision”) is typically made at the site of the bunion (e.g., the distal end of the affected metatarsal bone), and provides an access portal for cutting and realigning the bone. The other incision (e.g., “proximal incision”) is typically made near the proximal end of the affected metatarsal bone, providing an insertion trajectory for one or more fixation screws once the bone has been cut and realigned. In current practice, both the distal incision and the proximal incision are linear horizontal incisions in that they are generally parallel to a longitudinal axis of the foot (e.g., an imaginary axis extending through the foot in a heal-to-toe direction). As used herein, the term “generally parallel” is meant to include orientations that extend in the same general direction, if not strictly parallel.

### SUMMARY

In some embodiments, the intramedullary targeting guide assembly disclosed herein may include a clamp, a rail member, a drill guide assembly, a rotation assembly, and a vertical shift assembly. By way of example, the clamp (or “forceps clamp”) is configured to register the intramedullary targeting guide assembly to a bone by clamping to the cortical wall of the bone. More specifically, the clamp includes a first portion (e.g., intramedullary tip jaw) configured to extend at least partially into the intramedullary canal of the bone through an osteotomy opening and a second portion (e.g., shovel tip jaw) configured for positioning extramedullary but subcutaneously such that a portion of the cortical wall of the bone is situated between the first portion and the second portion. The clamp may then be actuated or “clamped” such that the first and second portions forcibly engage the cortical wall located therebetween to secure the clamp to the bone.

In some embodiments, the rail member may be a base to which the clamp, drill guide assembly and rotation assembly are attached. In some embodiments, the drill guide assembly includes a drill guide base and drill guide pivot and is positioned on the distal end of the rail member and configured to aid the user in (a) determining the desired trajectory of the bone anchor into the bone under repair and (b) positioning a guide wire into the bone under repair in the desired trajectory. In some embodiments, the rotation assembly includes a rotation guide, a rotation mount, and a horizontal shift screw, and enables a user to controllably rotate and/or push and/or pull a portion of the target area into position to ensure proper alignment of the bone fragments before anchor placement. By way of example, in a bunion repair surgery the rotation assembly may be used to rotate sesamoid bones located below the metatarsal head of the bone under repair. In some embodiments, the vertical shift assembly includes a vertical shift base, a vertical support paddle, and a vertical shift screw, and may be used to position and stabilize one side of bone fracture or osteotomy prior to fixation.

In some embodiments, in addition to the method of using the intramedullary targeting guide assembly in the course of a minimally invasive bunion surgery, this disclosure described several other novel methods. For example, this disclosure describes an example method of registering a targeting guide or other surgical instrument by clamping to a cortical wall without penetrating through the cortical wall. In some embodiments, this method is performed in the context of an osteotomy procedure or in a similar fracture scenario. In some embodiments, the method includes introducing a clamp to a surgical target site comprising a bone fracture such that a first jaw member of the clamp is inserted into an intramedullary canal of the fractured bone and a second jaw member is applied extramedullary such that a portion of cortical wall of the fractured bone is situated between the first jaw member and the second jaw member. In some embodiments, the second jaw member is introduced subcutaneously. In some embodiments, a force is applied to the first and second jaws to clamp the jaws together with the cortical bone therebetween, thereby securing the clamp to the cortical bone without creating additional holes in the bone. In some embodiments, the method further includes attaching a surgical instrument to the secured clamp. In some embodiments, the surgical instrument is a targeting guide.

In some embodiments, this disclosure describes a method of aligning a surgical instrument placed at least partially within bone using at least three radiographic markers to triangulate positioning under fluoroscopy in multiple planes.

In some embodiments, a surgical targeting guide system configured for in situ assembly is provided, comprising a clamp member, a rail member, a drill guide assembly, a rotation guide assembly, and a vertical shift assembly. In some embodiments, the clamp member defines a proximal end of the surgical targeting guide system and has a first elongated jaw member hingedly attached to a second elongated jaw member, a locking element configured to secure the first and second elongated jaw members in a clamped position, and a longitudinal axis and a transverse axis defining an alignment plane extending through the first and second elongated jaw members. In some embodiments, the rail member is configured for in situ coupling with the clamp member such that the rail member is coplanar with the alignment plane. In some embodiments, the drill guide assembly is configured for in situ coupling with the rail member, defines a distal end of the surgical targeting guide assembly, and comprises a coupling element, a housing, and a pivot member, the coupling element configured to removably couple the drill guide assembly to the rail member, the pivot member positioned within the housing and configured to pivot about a vertical axis perpendicular to the longitudinal axis, and the pivot member further comprising a central guide aperture configured to receive a guide wire therethrough. In some embodiments, the rotation guide assembly is configured for in situ slidable coupling with the rail member and includes a base member having at least one curved flange and a rotation mount slidably coupled with the at least one curved flange. In some embodiments, the vertical shift assembly is configured for in situ coupling with the rotation guide assembly and comprises a base member including a coupling element configured to removably couple the vertical shift assembly to the rotation guide assembly, a support paddle, and an actuator configured to effect displacement of the support paddle in a direction perpendicular to the alignment plane.

Additionally, the surgical targeting guide system described herein may be further defined by one or more of the following features: wherein the locking element of the clamp member comprises a ratchet mechanism or a spin lock mechanism; wherein the first jaw member has a tapered tip configured to facilitate insertion into an intramedullary canal of a bone; wherein the second jaw member has a shovel tip including a bone engagement portion configured to interact with extramedullary bone to prevent movement of the shovel tip relative to the extramedullary bone. ; wherein the rail member comprises an elongated coupling element configured to slideably engage the rotation guide assembly; wherein the rail member is coupled with the clamp by way of an interaction with an engagement feature on the second jaw member; wherein the drill guide assembly comprises a coupling element configured to removably couple the drill guide assembly to the rail member; wherein the drill guide assembly further comprises a locking element actuatable to lock the pivot member in a targeted position; wherein the rotation assembly includes a transverse coupling slot configured to slidably couple the with the elongated coupling element of the rail member; wherein the rotation assembly includes a transverse actuator configured to couple with a bone engagement element such that the bone engagement element is controllably adjustable upon actuation of the transverse actuator; wherein the rotation assembly further includes a locking element configured to lock the rotation mount in a position along the at least one curved flange; wherein the support paddle includes a visualization element configured to indicate a position of the support paddle under fluoroscopy; and/or wherein the visualization element comprises a through-hole.

In some embodiments, a method of aligning a surgical instrument is disclosed, comprising using a plurality of radiographic markers associated with the surgical instrument to triangulate a position under fluoroscopy in at least two planes, wherein first and second radiographic markers of the plurality of radiographic markers are used to verify positioning of the surgical instrument in a first plane, and wherein a third radiographic marker of the plurality of radiographic markers is used to verify positioning of the surgical instrument in a second plane, the second plane being different from the first plane.

Additionally, the method of aligning a surgical instrument disclosed herein may be further defined by one or more of the following features: wherein the first and second radiographic markers are positioned within a patient's body; wherein the first and second radiographic markers are configured to verify rotational alignment; wherein the third radiographic marker is positioned exterior to the patient's body; and/or wherein the third radiographic marker is configured to verify directional alignment.

In some embodiments, a method of registering a surgical instrument to a severed bone segment of a patient is provided, comprising the steps of: (a) introducing a clamp member to a surgical target site comprising the severed bone, the clamp member comprising a first jaw member hingedly coupled to a second jaw member; (b) inserting the first jaw member into an exposed intramedullary canal of the severed bone without penetrating the cortical wall of the severed bone; (c) positioning the second jaw member extramedullary such that a portion of the cortical wall of the severed bone is positioned between the first jaw member and the second jaw member; (d) applying a compressive force to the first and second jaw members to urge the first and second jaw members toward one another with the cortical bone positioned therebetween, securing the clamp member to the cortical bone without creating additional holes in the cortical bone; and (e) coupling a surgical instrument to the clamp member.

Additionally, the method of registering a surgical instrument to a severed bone segment of a patient may be further defined by one or more of the following features; wherein the surgical instrument is coupled to the clamp member after the clamp member has been secured to the cortical bone; wherein the surgical instrument is a targeting guide; wherein the step of coupling a surgical instrument to the clamp member occurs before the step of applying a compressive force to the first and second jaw members; wherein the severed bone segment is a result of at least one of an osteotomy procedure and a fracture event; and/or further comprising the step of adjusting the position of the clamp member after completing the steps of inserting the first jaw member and positioning the second jaw member.

### BRIEF DESCRIPTION OF THE DRAWINGS

Many advantages of the present disclosure will be apparent to those skilled in the art with a reading of this specification in conjunction with the attached drawings, wherein like reference numerals are applied to like elements and wherein:

FIGS. 1-3 are perspective views of an example of an intramedullary targeting guide assembly, according to some embodiments;

FIG. 4 is a top plan view of the intramedullary targeting guide assembly of FIG. 1, according to some embodiments;

FIG. 5 is a side plan view of the intramedullary targeting guide assembly of FIG. 1, according to some embodiments;

FIG. 6 is another side plan view of the intramedullary targeting guide assembly of FIG. 1, rotated 90° relative to the view shown in FIG. 5, according to some embodiments;

FIG. 7 is a lateral perspective view of an example of an intramedullary tip jaw of a forceps clamp forming part of the intramedullary targeting guide assembly of FIG. 1, according to some embodiments;

FIG. 8 is a medial perspective view of the intramedullary tip jaw of FIG. 7, according to some embodiments;

FIG. 9 is a lateral perspective view of an example of a shovel tip jaw of a forceps clamp forming part of the intramedullary targeting guide assembly of FIG. 1, according to some embodiments;

FIG. 10 is a medial perspective view of the shovel tip jaw of FIG. 9, according to some embodiments;

FIGS. 11-12 are perspective views of an example of a rail member forming part of the intramedullary targeting guide assembly of FIG. 1, according to some embodiments;

FIG. 13 is a side plan view of the rail member of FIG. 11, according to some embodiments;

FIG. 14 is a bottom plan view of the rail member of FIG. 11, according to some embodiments;

FIGS. 15-16 are perspective views of an example of a drill guide base forming part of the intramedullary targeting guide assembly of FIG. 1, according to some embodiments;

FIG. 17 is a side plan view of the drill guide base of FIG. 15, according to some embodiments;

FIG. 18 is a perspective view of an example of a drill guide pivot forming part of the intramedullary targeting guide assembly of FIG. 1, according to some embodiments;

FIG. 19 is a perspective view of an example of a thumb screw forming part of the intramedullary targeting guide assembly of FIG. 1, according to some embodiments;

FIGS. 20-21 are perspective views of an example of a rotation guide forming part of the intramedullary targeting guide assembly of FIG. 1, according to some embodiments;

FIGS. 22-23 are perspective views of an example of a rotation mount forming part of the intramedullary targeting guide assembly of FIG. 1, according to some embodiments;

FIGS. 24-25 are perspective views of an example of a horizontal shift screw forming part of the intramedullary targeting guide assembly of FIG. 1, according to some embodiments;

FIGS. 26-27 are perspective views of an example of a vertical shift base forming part of the intramedullary targeting guide assembly of FIG. 1, according to some embodiments;

FIG. 28 is a perspective view of an example of a vertical support paddle forming part of the intramedullary targeting guide assembly of FIG. 1, according to some embodiments;

FIGS. 29-30 are perspective views of an example of a vertical shift screw forming part of the intramedullary targeting guide assembly of FIG. 1, according to some embodiments;

FIG. 31 is a flowchart depicting several steps in an example method of in situ assembly and use of the intramedullary targeting guide assembly of FIG. 1 during a bunion surgery, according to some embodiments;

FIG. 32 is a fluoroscopy scan image depicting an example of one step in the method of FIG. 31, in particular the step of performing an osteotomy in a target bone segment to create an opening into the intramedullary canal of the severed bone segment, according to some embodiments;

FIGS. 33-35 are a series of illustrations depicting an example of another step in the method of FIG. 31, in particular the step of introducing a clamp member forming part of the intramedullary targeting guide of FIG. 1 to the surgical site, for example by inserting an intramedullary tip jaw directly into the intramedullary canal of the severed bone segment, according to some embodiments;

FIG. 36 is a scan image depicting an example of one step in the method of FIG. 31, in particular the step of moving the clamp into position on plane in the middle of the severed bone segment, and more particularly illustrating proper rotational alignment of the clamp member within the intramedullary canal of the severed bone segment, according to some embodiments;

FIG. 37 is a fluoroscopy scan image depicting another example of the step of FIG. 36, particularly illustrating an improper rotational alignment of the clamp member within the intramedullary canal of the severed bone segment, according to some embodiments;

FIG. 38 is an illustration depicting a fluoroscopy scan image showing another example of the step of FIG. 36, particularly illustrating a proper directional alignment of the clamp member within the intramedullary canal of the severed bone segment, according to some embodiments;

FIGS. 39-40 are illustrations depicting a fluoroscopy scan image showing another example of the step of FIG. 36, particularly illustrating improper directional alignments of the clamp member within the intramedullary canal of the severed bone segment, according to some embodiments;

FIG. 41 is an illustration and fluoroscopic scan image presented side-by-side and illustrating another example of one step in the method of FIG. 31, in particular the step of applying a clamping force on the severed bone segment such that the cortical wall is captured and held between the intramedullary tip jaw and the shovel tip jaw of the clamp member, according to some embodiments;

FIG. 42 is a fluoroscopic scan image depicting an example of an optional step in the method of FIG. 31, in particular the step of inserting a security wire along the intramedullary tip jaw and into the cortical wall of the base of the metatarsal bone, according to some embodiments;

FIG. 43 illustrates another example of one step in the method of FIG. 31, in particular the step of coupling the rotational assembly of the intramedullary targeting guide of FIG. 1 to the rail member of FIG. 11, according to some embodiments;

FIG. 44 is a fluoroscopic scan image depicting another example of a step in the method of FIG. 31, in particular the step of adjusting the medial-lateral position of the vertical support paddle of the intramedullary targeting guide of FIG. 1 such that a radiographic marker is positioned in a desired location relative to the severed bone segment, according to some embodiments;

FIG. 45 is a fluoroscopic scan image depicting another example of a step in the method of FIG. 31, in particular the step of adjusting the vertical position of the vertical support paddle so that the fragments of the severed bone segment are in alignment, according to some embodiments;

FIGS. 46-49 are a series of illustrations and fluoroscopic images depicting another example of a step in the method of FIG. 31, in particular the step of inserting a stabilization wire through the rotation mount and into the middle of the distal fragment of the severed bone segment, according to some embodiments;

FIG. 50-51 illustrate another example of one step in the method of FIG. 31, in particular the step of rotating the sesamoids into position by using the rotation guide, according to some embodiments;

FIG. 52 illustrates another example of one step in the method of FIG. 31, in particular the step of stabilizing the distal portion of the intramedullary targeting guide assembly by inserting a stabilizing element through a horizontal slot of the drill guide base and into a bone segment that is not the bone under repair to secure the distal portion of the intramedullary targeting guide assembly in place, according to some embodiments;

FIG. 53 illustrates another example of one step in the method of FIG. 31, in particular the step of determining the location of the proximal incision, according to some embodiments;

FIGS. 54-57 illustrate another example of one step in the method of FIG. 31, in particular the step of determining the insertion trajectory and placing the insertion wire into the target bone segment, according to some embodiments;

FIGS. 58-60 illustrate another example of one step in the method of FIG. 31, in particular the step of removing the various positioning wires and the drill guide assembly, according to some embodiments;

FIG. 61 illustrates another example of one step in the method of FIG. 31, in particular the step of advancing a drill guide over the insertion wire to the proximal incision, according to some embodiments;

FIGS. 62-64 illustrate another example of one step in the method of FIG. 31, in particular the step of removing the remainder of the intramedullary targeting guide assembly from the target site, according to some embodiments;

FIGS. 65-66 illustrate top and side views, respectively, of an example of the final screw placement after using the intramedullary targeting guide assembly of FIG. 1, according to some embodiments.

FIGS. 67-69 illustrate removal of a piece of bone during the bunion procedure, according to some embodiments; and

FIG. 70 is a flowchart describing an example method of registering a surgical instrument to a severed bone segment of a patient by clamping to a cortical wall without penetrating through the cortical wall.

### DETAILED DESCRIPTION OF A PREFERRED EMBODIMENT

Illustrative embodiments of the disclosure are described below. In the interest of clarity, not all features of an actual implementation are described in this specification. It will of course be appreciated that in the development of any such actual embodiment, numerous implementation-specific decisions must be made to achieve the developers'specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure. The intramedullary targeting guide assembly and related methods disclosed herein boasts a variety of inventive features and components that warrant patent protection, both individually and in combination.

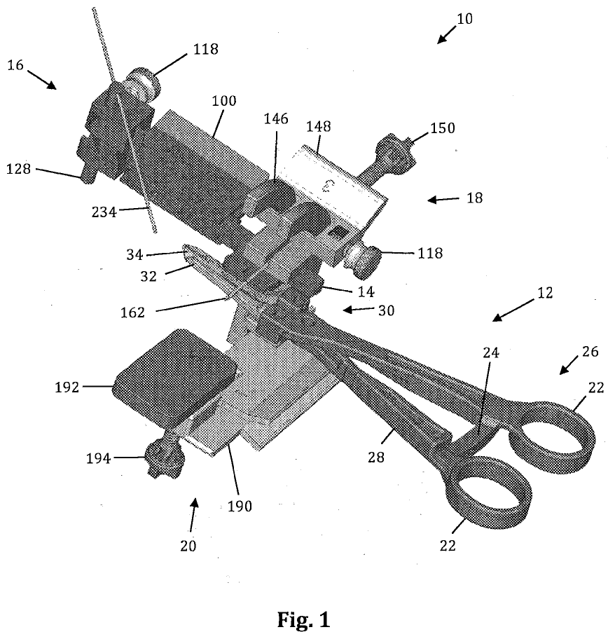

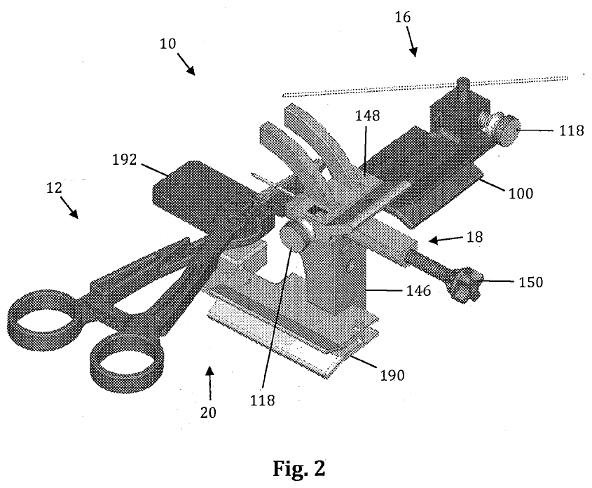

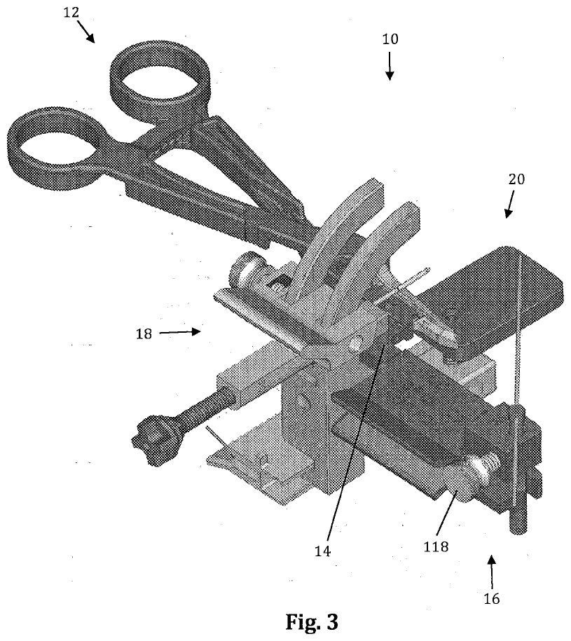

FIGS. 1-6 illustrate an example of an intramedullary targeting guide assembly 10 according to some embodiments of the disclosure. In some embodiments, the intramedullary targeting guide assembly 10 may include a clamp 12, a rail member 14, a drill guide assembly 16, a rotation assembly 18, and a vertical shift assembly 20. By way of example, the clamp 12 (or “forceps clamp 12”) is configured to register the intramedullary targeting guide assembly 10 to a bone by clamping to the cortical wall of the bone. More specifically, the clamp 12 includes a first portion (e.g., intramedullary tip jaw 32) configured to extend at least partially into the intramedullary canal of the bone through an osteotomy opening or other fracture and a second portion (e.g., shovel tip jaw 34) configured for positioning extramedullary but subcutaneously (for example) such that a portion of the cortical wall of the bone is situated between the first portion and the second portion. The clamp 12 may then be actuated or “clamped” such that the first and second portions forcibly engage the cortical wall located therebetween to secure the clamp 12 to the bone.

In some embodiments, the rail member 14 may be a base to which the clamp 12, drill guide assembly 16 and rotation assembly 18 are attached. In some embodiments, the drill guide assembly 16 includes a drill guide base 100 and drill guide pivot 128 and is positioned on the distal end of the rail member 12 and configured to aid the user in (a) determining the desired trajectory of the bone anchor into the bone under repair and (b) positioning a guide wire into the bone under repair in the desired trajectory. In some embodiments, the rotation assembly 18 includes a rotation guide 146, a rotation mount 148, and a horizontal shift screw 150, and enables a user to controllably rotate and/or push and/or pull a portion of the target area into position to ensure proper alignment of the bone fragments before anchor placement. By way of example, in a bunion repair surgery the rotation assembly 18 may be used to rotate sesamoid bones located below the metatarsal head of the bone under repair. In some embodiments, the vertical shift assembly 20 includes a vertical shift base 190, a vertical support paddle 192, and a vertical shift screw 194, and may be used to position and stabilize one side of bone fracture or osteotomy prior to fixation.

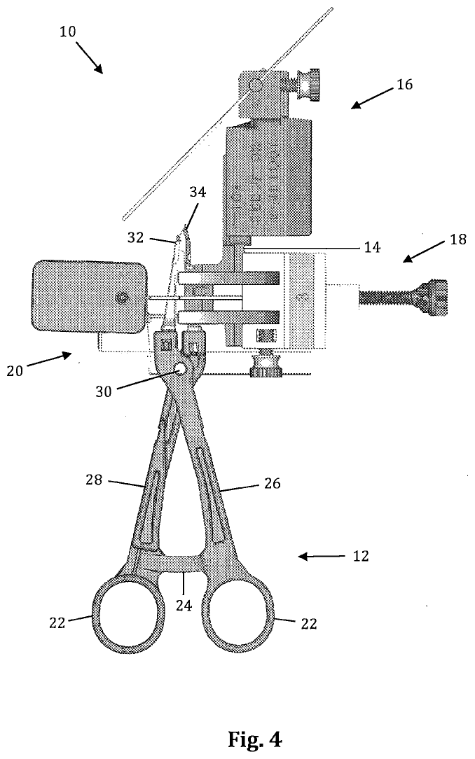

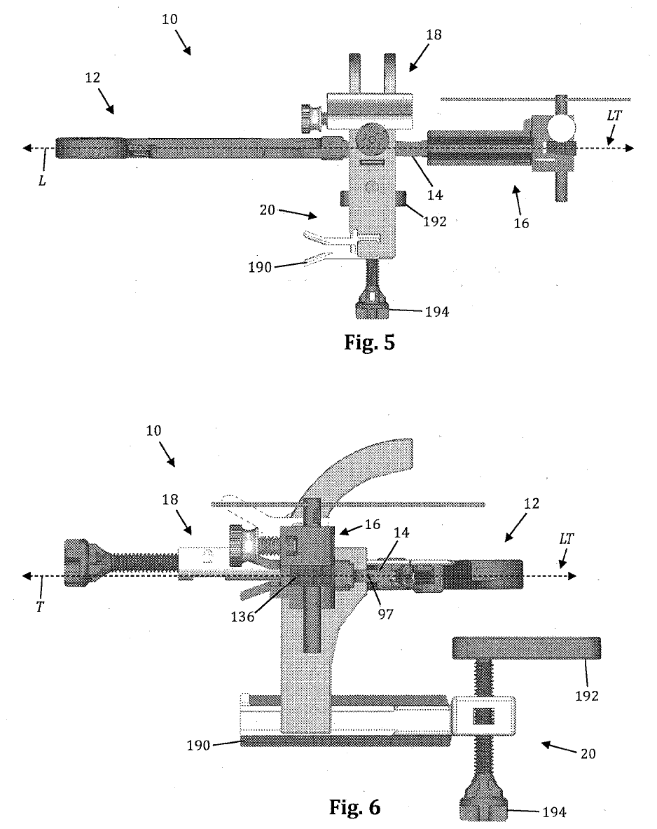

In some embodiments, the drill guide assembly 10 has longitudinal axis L and a transverse axis T that define an alignment plane LT, as shown by way of example in FIGS. 5-6. In some embodiments, the components of the drill guide assembly 10 are assembled such that the clamp 12 and the rail member 14 are coplanar with alignment plane LT. In some embodiments, any of the various components of the drill guide assembly 10 may be made from a radiolucent material to enable fluoroscopic position verification from multiple directions, including but not limited to anterior-posterior and medial-lateral.

In some embodiments, the clamp 12 comprises a forceps-style clamp 12 having a ring handle 22, locking element 24, first shank 26, second shank 28, a fulcrum 30, an intramedullary tip jaw 32, and a shovel tip jaw 34. By way of example, the ring handle 22 is located at a proximal end 36 of the clamp 12 and is configured for manipulation by a user to open the jaw at the distal end 38 of the clamp 12, for example by forcing the ring handles apart (to open) and by forcing the ring handles 22 toward one another (to close) the jaw at the distal end 38. In some embodiments, the locking element 24 comprises a ratchet mechanism including a pair of overlapping flanges extending toward one another from each of the first and second shanks 26, 28. By way of example, the overlapping extensions each have ratchet teeth that interact with one another to enable translation of the flanges toward one another but prohibit translation of the flanges away from one another and maintain a forcible clamping of the jaws. In some embodiments, the locking element 24 may comprise a spin lock. In some embodiments, the first and second shanks 26, 28 are pivotably connected to one other at the fulcrum 30. In some embodiments, the intramedullary tip jaw 32 is coupled to the distal end of the first shank 26, and the shovel tip jaw 34 is coupled with the distal end of the second shank 28.

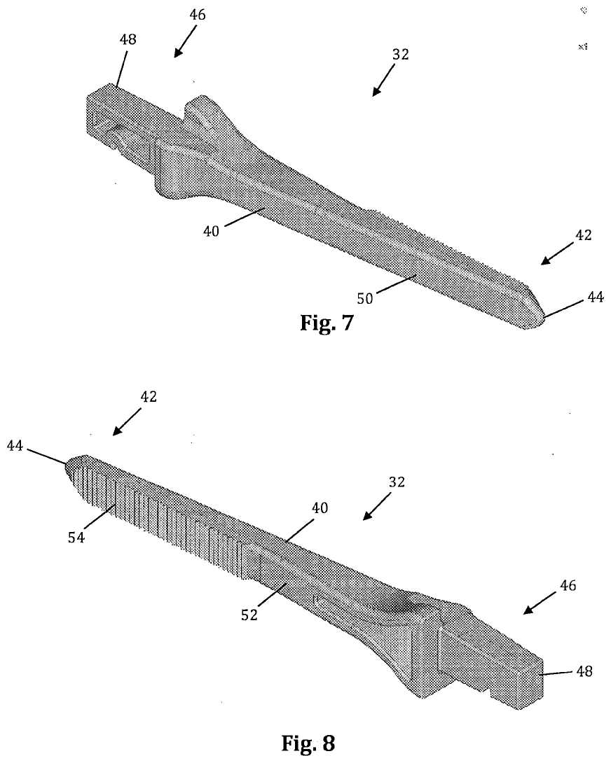

Referring to FIGS. 7-8, by way of example the intramedullary tip jaw 32 comprises an elongated body 40 having a distal end 42 including a tapered tip 44 with a smooth surface configured to facilitate advancement of the intramedullary tip jaw 32 into bone, and a proximal end 46 including a coupling feature 48 configured to securely couple the intramedullary tip jaw 32 to the first shank 26. In some embodiments, the intramedullary tip jaw 32 has a smooth lateral surface 50 and a medial facing surface 52 including a textured or ridged portion 54 configured to interact with bone to prevent movement of the intramedullary tip jaw 32 relative to the contacted bone when the clamp 12 is in a closed or clamped orientation. By way of example, in a minimally invasive bunion surgery, the intramedullary tip jaw 32 may be inserted directly into the intramedullary canal of the first metatarsal bone.

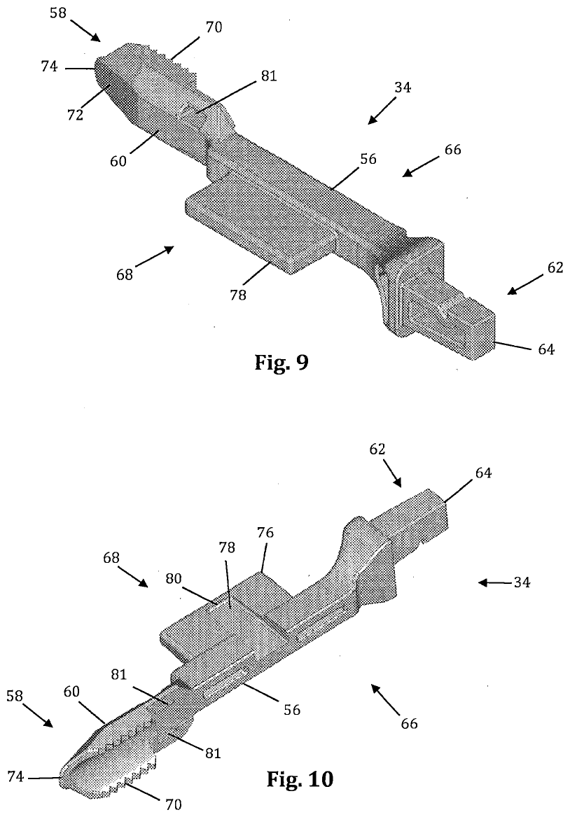

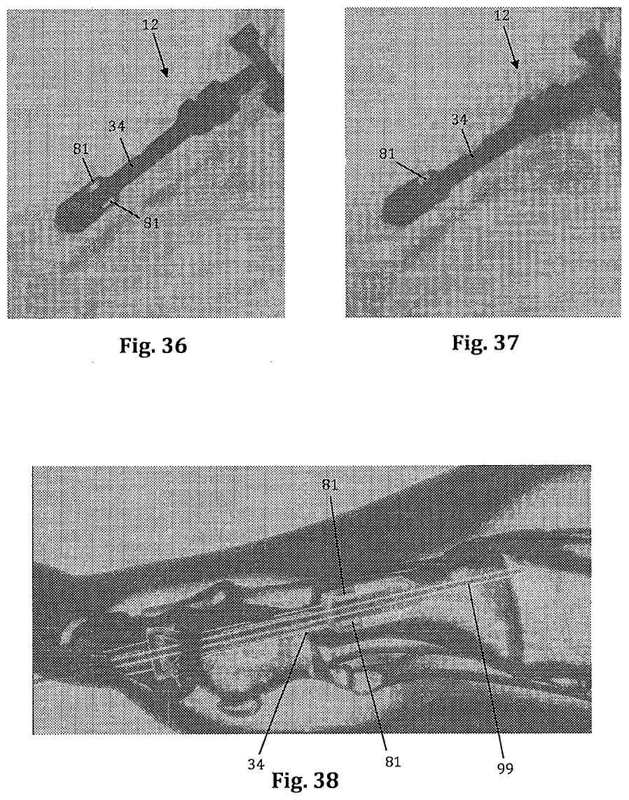

Referring to FIGS. 9-10, by way of example the shovel tip jaw 34 comprises an elongated body 56 having a distal end 58 including a shovel tip 60 and a proximal end 62 including a coupling feature 64 configured to securely couple the shovel tip jaw 34 to the to the second shank 28. By way of example, the shovel tip jaw 34 has a medial side 66 and a lateral side 68. In some embodiments, the shovel tip 60 includes a textured or ridged portion 70 on the medial side configured to interact with bone to prevent movement of the shovel tip jaw 34 relative to the contacted bone when the clamp 12 is in a closed or clamped orientation. In some embodiments, the shovel tip 60 includes a smooth lateral surface 72 and a tapered tip 74 configured to facilitate subcutaneous introduction into the patient. In some embodiments, the shovel tip jaw 34 further includes a coupling flange 76 on the lateral side of the elongated body 56, the coupling flange 76 configured to interact with the clamp engagement feature 90 of the rail member 14 as described below. In some embodiments, the coupling flange 76 has a central recess 78 configured to slideably engage the deflectable flange 96 of the rail member 14 to facilitate secure engagement of the coupling flange 76 to the rail member 14. In some embodiments, the central recess 78 has an end wall 80 configured to interact with the deflectable flange 96 of the rail member 14 to provide a locking interface between the rail member 14 and the shovel tip jaw 34. By way of example, in a minimally invasive bunion surgery described herein, the shovel-tip jaw 34 may contact the first metatarsal bone extramedullary but subcutaneously. In some embodiments, the shovel tip jaw 34 may further include one or more radiographic markers that help enable a surgeon to verify or adjust positioning of the clamp 12 in real time under fluoroscopy during a surgical procedure. By way of example, the shovel tip jaw 34 may include distal slots 81 that are visible under fluoroscopy (as an absence of material) and which a surgeon can use to verify and/or adjust position of the clamp 12. (See, e.g., FIGS. 36-37).

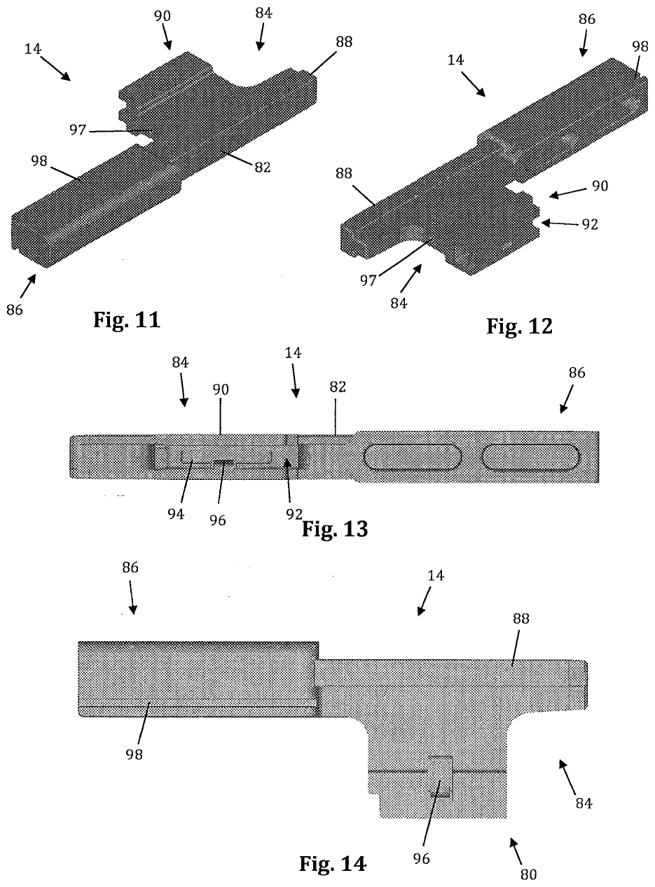

Referring to FIGS. 11-14, in some embodiments the rail member 14 comprises an elongated body 82 having a proximal portion 84 and a distal portion 86. In some embodiments, the proximal portion 84 has an elongated coupling element 88 configured to slideably engage the coupling aperture 158 of the rotation guide 146 of the rotation assembly 18, as shown in FIG. 1. In some embodiments, the rotation assembly 18 may translate along the elongated coupling element 88 of the rail member 14 to ensure proper positioning of the rotation assembly 18 during use. In some embodiments, the elongated coupling element 88 may have a cross-sectional shape configured to allow for translation only while preventing any pivoting or rocking about the rail member 14. By way of example, the elongated coupling element 88 of the embodiment shown in FIGS. 11-14 has a T-shaped cross-section, however other shapes are possible.

In some embodiments, the proximal portion 84 further includes a clamp engagement feature 90 extending laterally from the elongated body 82 opposite the elongated coupling element 88 and configured to securely couple the rail member 14 to the clamp 12 such that the rail member 14 is coplanar with the clamp 12 within the alignment plane LT. By way of example only, the clamp engagement feature 90 includes an elongated channel 92 oriented parallel to a longitudinal axis of the rail member 14. In some embodiments, the elongated channel 92 is sized and configured to receive at least a portion of the elongated body 56 of the shovel tip jaw 34 therein. In some embodiments, the clamp engagement feature 90 further includes a transverse recess 94 including a deflectable flange 96 positioned therein. By way of example, the transverse recess 94 may be sized and configured to snugly receive the coupling flange 76 of the shovel tip jaw 34 therein such that the deflectable flange 96 is at least partially situated in the central recess 78 of the coupling flange 76. In some embodiments, upon full insertion of the coupling flange 76 into the transverse recess 94, the deflectable flange 96 will deflect over the end wall 80 of the central recess 78 and snap back into place, thereby preventing decoupling of the rail member 14 and the shovel tip jaw 34 absent sufficient applied force. Additionally, in some embodiments the snapping of the deflectable flange 96 may provide audio and/or tactile feedback to communicate to the use that the rail member 14 has been fully secured to the clamp 12.

In some embodiments, the rail member 14 may further include an elongated guide aperture 97 oriented parallel to the longitudinal axis of the rail member 14. By way of example, the elongated guide aperture 97 is configured to enable passage of a guide wire (e.g., external guide wire 99) that may be used in the initial positioning of the clamp 12 within the intramedullary canal to ensure that the intramedullary tip jaw 32 extends into the middle of the intramedullary canal before clamping. (See, e.g., FIG. 38). In some embodiments, the external guide wire 99 passing through guide aperture 97 may also be used to help determine the location of the proximal incision during use in a bunion repair procedure. In some embodiments, the elongated guide aperture 97 extends longitudinally through the alignment plane LT (See, e.g., FIG. 6) such that any guide wire 99 inserted through the elongated guide aperture 97 is positioned coplanar with the alignment plane LT extending through the rail member 14 and the clamp 12.

In some embodiments, the distal portion 86 of the rail member 14 is configured to engage the drill guide assembly 16. In some embodiments, the distal portion 86 includes one or more elongated recesses 98 configured to receive the vertical flanges 124 of the coupling element 104 of the drill guide base 100 to securely couple the drill guide assembly 16 to the rail member 14.

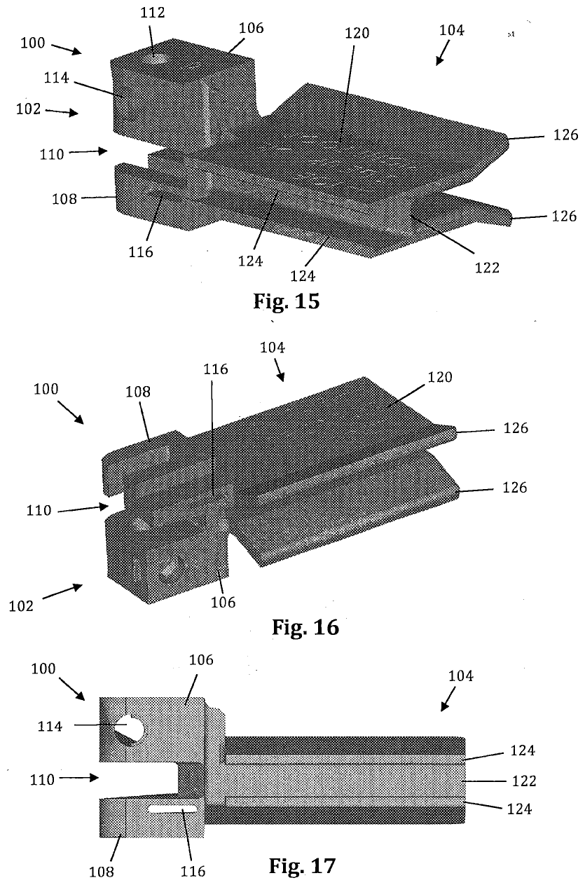

FIGS. 15-17 illustrate an example of a drill guide base 100 forming part of the drill guide assembly 16, according to some embodiments of the disclosure. In some embodiments, the drill guide base 100 comprises a pivot housing 102 and a coupling element 104. By way of example, the pivot housing 102 includes an upper housing 106 separated from a lower stabilizer 108 by a gap 110. In some embodiments, the upper housing 106 may have a vertical aperture 112 extending vertically completely through the upper housing 106 and a transverse aperture 114 extending completely through the upper housing 106 in a transverse orientation such that the vertical aperture 112 and transverse aperture 114 intersect within the interior of the upper housing 106. In some embodiments, the vertical aperture 114 is sized and configured to receive a pivot post 132 of the drill guide pivot 128 therein such that the pivot post 132 is rotatable within the vertical aperture 114. In some embodiments, the transverse aperture 114 is at least partially threaded and configured to receive the threaded shank 142 of a thumb screw 118 therein (FIG. 19), which may be actuated to lock or unlock the rotational position of the drill guide pivot 128. In some embodiments, the gap 110 is sized and configured to receive the pivot block 130 therein such that the pivot block 130 may rotate within a single plane within the gap to enable the user to select the desired anchor trajectory through the bone under repair. In some embodiments, the lower stabilizer 108 may include a horizontal slot 116 formed therein and configured to receive a stabilizing element (e.g., K-wire, pin, etc.) that passes through the horizontal slot 116 and into a bone segment that is not the bone under repair to secure the distal portion of the intramedullary targeting guide assembly 10 in place.

In some embodiments, the coupling element 104 may be any feature that securely and immovably couples the drill guide assembly 16 to the rail member 14. In some embodiments, the coupling element 104 may be configured for easy removal during the procedure. In some embodiments, the coupling element 104 may comprise a pair of rectangular (for example) panels 120 spaced apart by a vertical separator 122 that divides the coupling element 104 into a medial portion and a lateral portion. In some embodiments, the medial portion comprises a pair of opposing elongated vertical flanges 124 positioned along the medial edge of each of the panels 120 and oriented such that the vertical flanges 124 extend toward one another. By way of example, the vertical flanges 124 are configured to engage the elongated recesses 98 of the rail member 14 to securely couple the drill guide assembly 16 to the rail member 14. In some embodiments, the lateral portion of the panels 120 each include a handle member 126 that may comprise oblique flanges or similar suitable user interface structure configured to enable a user to push toward one another thereby causing the vertical flanges 124 to move away from each other (e.g., by pivoting or deforming about the vertical separator 122 which acts as a fulcrum) and disengage from the elongated recesses 98 of the rail member 14 for easy repositioning (e.g., by sliding along the rail member) and/or removal of the drill guide assembly 16 during use.

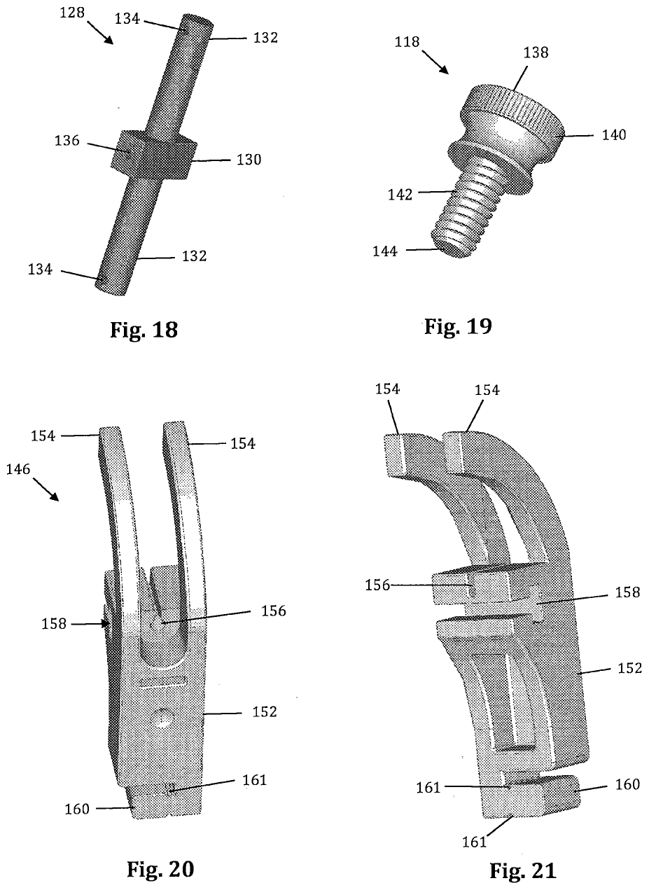

Referring to FIG. 18, in some embodiments the drill guide pivot 128 includes a pivot block 130 and a pair of pivot posts 132 extending vertically from the pivot block 130. In some embodiments, the pivot block 130 has planar upper and lower surfaces and is configured to pivot within the gap 110 in a single plane (e.g., the alignment plane LT). In some embodiments, the upper pivot post 132 is configured to extend through the vertical aperture 112 of the upper housing 106 and the lower pivot post 132 is configured to extend through the lower stabilizer 108. In some embodiments, each of the pivot posts 132 includes a guide aperture 134 positioned near the outer ends of the pivot posts 132 (e.g., farthest away from the pivot block 130) and extending transversely through the pivot post 132. By way of example, the guide apertures 134 are configured to receive a guide wire (e.g., blunt-tip K-wire or template wire 234) therethrough. In some embodiments, the pivot block 130 includes a central guide aperture 136 extending transversely therethrough and configured to receive a guide wire (e.g., pointed-tip K-wire or insertion wire 236) therein. By way of example, in the example method described below, the guide wire extending through the guide aperture 134 of the upper pivot post 132 may be used as a template wire to assist the user in determining the desired trajectory through the bone under repair (e.g., using fluoroscopy to visualize the position of the wire), and the guide wire extending through the central guide aperture 136 within the pivot block 130 may be used to guide placement of a bone anchor during surgery. In some embodiments, the central guide aperture 136 extends longitudinally through the alignment plane LT (See, e.g., FIG. 6) such that any guide wire (e.g., a targeting wire) inserted through the central guide aperture 136 is positioned coplanar with the alignment plane LT extending through the rail member 14 and the clamp 12 (and the intramedullary canal) such that an inserted targeting wire is in plane with the target bone segments.

Referring to FIG. 19, in some embodiments the thumb screw 118 includes a head 138 including a textured surface 140 for improved grip for a user, and a threaded shank 142 configured for insertion into the transverse aperture 114 of the upper housing 106. By way of example, the threaded shank 142 comprises a distal interface surface 144 configured to contact the upper pivot post 132 of the drill guide pivot to lock the drill guide pivot 128 in position by compression and/or friction. For example, rotation of the thumb screw 118 in a clockwise direction advances the distal interface surface 144 into contact with the pivot post 132, locking the drill guide pivot 128 in position, which rotation of the thumb screw 118 in a counterclockwise direction retreats the distal interface surface 144 from the pivot post 132, thereby unlocking the drill guide pivot 128.

Referring now to FIGS. 20-21, the rotation guide 146 includes a vertically oriented base member 152, one or more curved flanges 154, a transverse wire slot 156, a coupling aperture 158, and a lower engagement member 160. In some embodiments, the curved flanges 154 are curved in a vertical-medial direction and are configured to interact with the rotation mount 148 such that the rotation mount can translate along the one or more curved flanges 154, as described below. By way of example, the transverse wire slot 156 is configured to enable passage of a transverse wire 162 (See, e.g., FIG. 1) during initial insertion of the wire 162 and before any translation of the rotation mount 148. By way of example, during rotation of the rotation mount 148, the transverse wire 162 may pass through the upper opening of the slot 156.

By way of example, the coupling aperture 158 is configured to slideably receive the elongated coupling element 88 of the rail member 14, as shown in FIG. 1. In some embodiments, the rotation assembly 18 may translate along the elongated coupling element 88 of the rail member 14 to ensure proper positioning of the rotation assembly 18 during use. In some embodiments, the coupling aperture 158 may have a cross-sectional shape configured to allow for translation only while preventing any pivoting or rocking of the rotation assembly 18 about the rail member 14. By way of example, the coupling aperture 158 of the embodiment shown in FIGS. 20-21 has a T-shaped cross-section, however other shapes are possible. In any event, the coupling aperture 158 will have a complementary shape to the elongated coupling element 88 of the rail member 14 to ensure a stable coupling. In some embodiments, the lower engagement member 160 includes one or more elongated recesses 161 and is configured to engage the coupling element 198 of the vertical shift assembly 20 as described below.

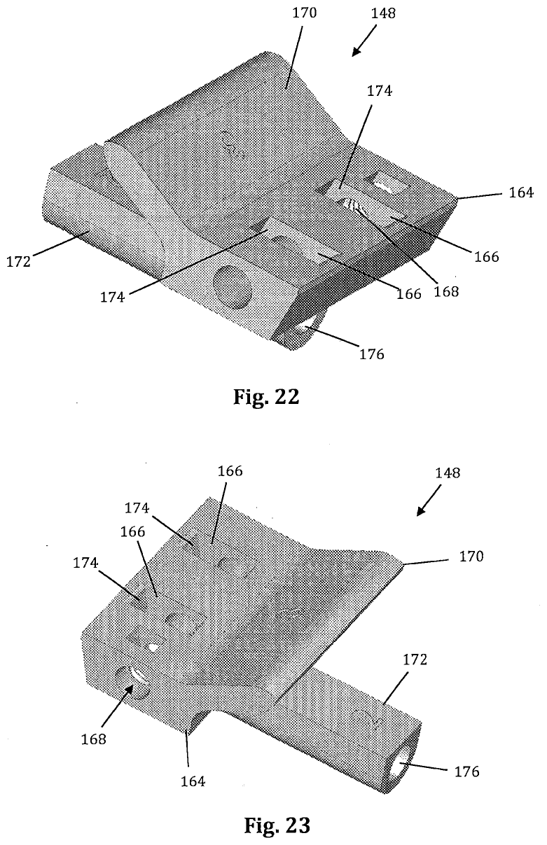

Referring to FIGS. 22-23, the rotation mount 148 includes a body portion 164 including at least one translation aperture 166 extending vertically therethrough, a lateral threaded aperture 168, a user interface flange 170, and a stabilization wire housing 172. In some embodiments, the body portion 164 includes a complimentary number of translation apertures 166 to match the number of curved flanges 154 on the rotation guide 146. By way of example, the translation apertures may have internal curved surfaces 174 having a curvature corresponding to the curvature of the curved flanges 154 to enable smooth migration of the rotation mount 148 along the curved flanges 154. In some embodiments, the lateral threaded aperture 168 intersects with one of the translation apertures 166 and is configured to receive a thumb screw 118 therein (FIG. 1) to enable locking and unlocking of the rotation mount 148 relative to the rotation guide 146 to secure or adjust the positioning of the rotation mount 148. By way of example, the user interface flange 170 may be an oblique flange or similar and provides the user with a handle for easier manipulation of the rotation mount 148 during use. By way of example, the transverse wire housing 172 includes a lumen 176 that may be at least partially threaded and is configured to enable passage of the transverse wire 162 through the rotation mount 148 and is further configured to threadedly mate with the horizontal shift screw 150.

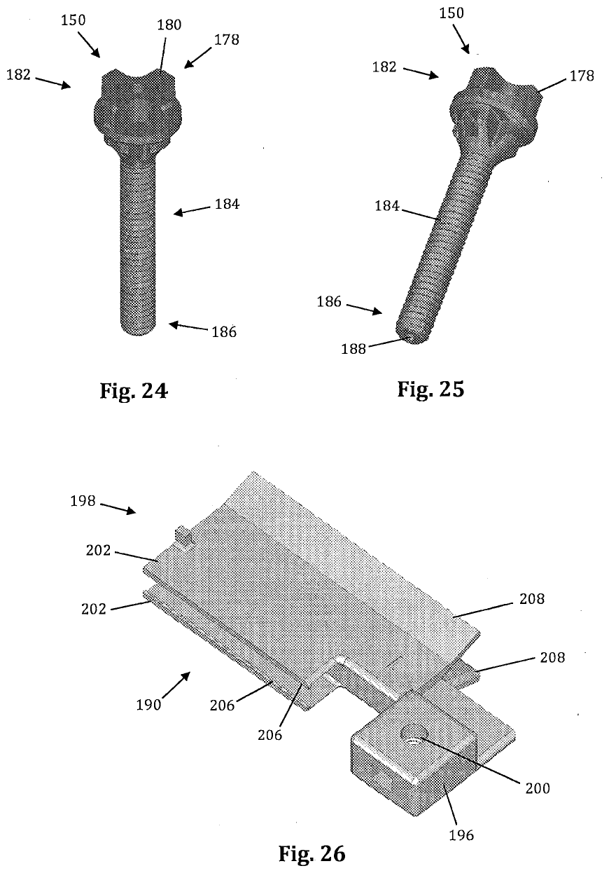

Referring to FIGS. 24-25, the horizontal shift screw 150 includes a head 178 including a user engagement feature 180 at a proximal end 182, an elongated threaded shaft 184 extending distally from the head 178 to a distal end 186. By way of example, the threaded shaft 184 includes a cannulation 188 extending proximally from the distally end at least partially into the shaft 184. In some embodiments, the cannulation 188 does not extend to the proximal end 182. In some embodiments, the cannulation 188 extends completely through the shaft 184.

In use, the horizontal shift screw 150 may be preloaded onto the rotation mount 148 by way of a threaded engagement with the lumen 176 of the stabilization wire housing 172. The horizontal shift screw 150 may be removed and the transverse wire 162 inserted as described below. In some embodiments, after insertion of the transverse wire 162, the horizontal shift screw 150 may be threaded into the lumen 176, with the proximal end of the transverse stabilization wire 162 positioned within the cannulation 188 to stabilize and protect the transverse wire during use. In use, the transverse wire 162 is inserted into the distal fragment. The horizontal shift screw 150 may then be coupled with the transverse wire 162 and used to controllably adjust the positioning of the distal fragment so that it is in position to receive the bunion fixation screw.

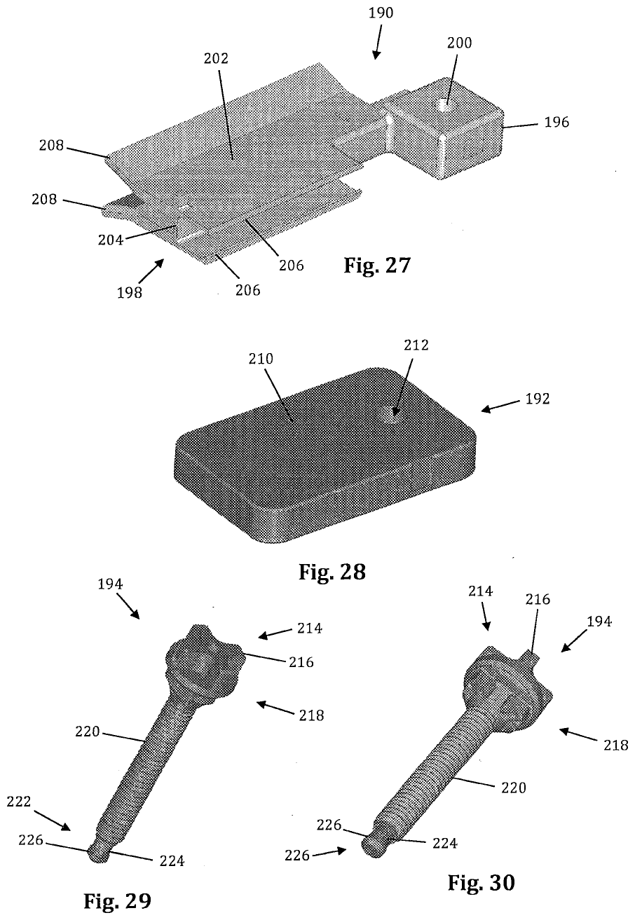

In some embodiments, the vertical shift assembly 20 includes a vertical shift base 190, a vertical support paddle 192, and a vertical shift screw 194, and may be used to position and stabilize one side of bone fracture or osteotomy prior to fixation. FIGS. 26-27 illustrate an example of a vertical shift base 190 forming part of the vertical shift assembly 20, according to some embodiments of the disclosure. In some embodiments, the vertical shift base 190 comprises a screw housing 196 and a coupling element 198. By way of example, the screw housing 196 includes a vertical aperture 200 extending vertically completely through the screw housing 196. In some embodiments, the vertical aperture 200 is at least partially threaded and configured to receive the threaded shank 220 of the vertical shift screw 194 therein, which may be actuated to raise or lower the vertical support paddle 192 as needed.

In some embodiments, the coupling element 198 may be any feature that securely couples the vertical shift assembly 20 to the rotation guide 146. In some embodiments, the coupling element 198 may be configured for easy adjustment during the procedure if a need arises to adjust the medial-lateral position of the vertical support paddle 192. In some embodiments, the coupling element 198 may comprise a pair of rectangular (for example) panels 202 spaced apart by a vertical separator 204 that divides the coupling element 198 into a medial portion and a lateral portion. In some embodiments, the medial portion comprises a pair of opposing elongated vertical flanges 206 positioned along the medial edge of each of the panels 202 and oriented such that the vertical flanges 206 extend toward one another. By way of example, the vertical flanges 206 are configured to engage the elongated recesses 161 of the lower engagement member 160 to securely couple the vertical shift assembly 20 to the rotation guide assembly 18. In some embodiments, the lateral portion of the panels 202 each include a handle member 208 that may comprise oblique flanges or similar suitable user interface structure configured to enable a user to push toward one another thereby causing the vertical flanges 206 to move away from each other (e.g., by pivoting or deforming about the vertical separator 204 which acts as a fulcrum) and disengage from the elongated recesses 161 of the lower engagement member 208 to enable medial-lateral adjustment of the vertical support paddle 192 during use.

Referring to FIG. 28, in some embodiments, the vertical support paddle 192 may have any shape capable of supporting a bone segment and/or body part, including but not limited to rectangular (as shown), square, circular, oval, or polygonal. In some embodiments, the vertical support paddle 192 may have a smooth upper surface 210 configured to contact patient anatomy and a through-hole 212 configured to receive the distal end 222 of the vertical shift screw 194 therein. In some embodiments, the connection between the vertical support paddle 192 and vertical shift screw 194 is such that the vertical support paddle 192 may rotate freely about the distal end 222 of the vertical shift screw 194, particularly when the vertical shift screw 194 is rotating. Thus, the vertical support paddle 192 may remain in a particular orientation when in contact with a patient anatomy while the vertical positioning is adjusted by rotation of the vertical shift screw 194. In some embodiments, the vertical support paddle 192 may be made of material that is at least partially viewable under fluoroscopy so that a user may determine the position of the vertical support paddle 192 in real time during a surgical procedure. In some embodiments, the through-hole 212 and/or vertical shift screw 194 coupled with the through-hole 212 (and/or a separate visualization element) may appear as a circular dot when viewed under fluoroscopy, which may be used as a guide to properly position the vertical support paddle 192 in a desired position. For example, in a bunion surgery, vertical support paddle 212 may be positioned such that the circular dot is aligned with the center of the distal fragment of the metatarsal bone under repair.

Referring to FIGS. 29-30, in some embodiments, the vertical shift screw 194 includes a head 214 including a user engagement feature 216 at a proximal end 218, an elongated threaded shank 220 extending distally from the head 214 to a distal end 222. By way of example, the distal end 222 may include a smooth cylindrical surface 224 that enables rotation of the vertical support paddle 192 about the vertical shift screw 194, and a distal ridge or lip 226 configured to interact with a complimentary lip or ridge within the through-hole 212 of the vertical support paddle 192 to enable secure coupling of the vertical shift screw 194 and the vertical support paddle 192.

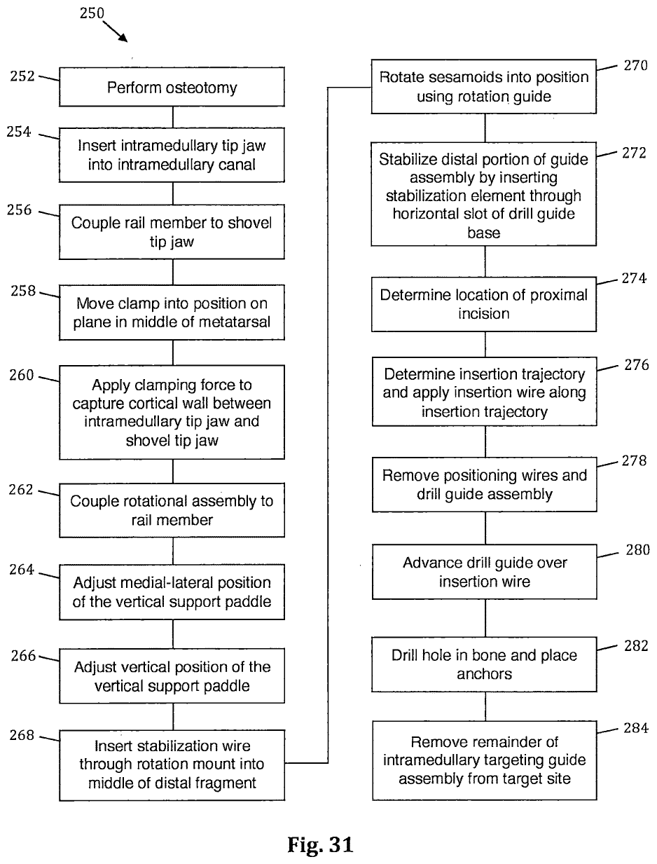

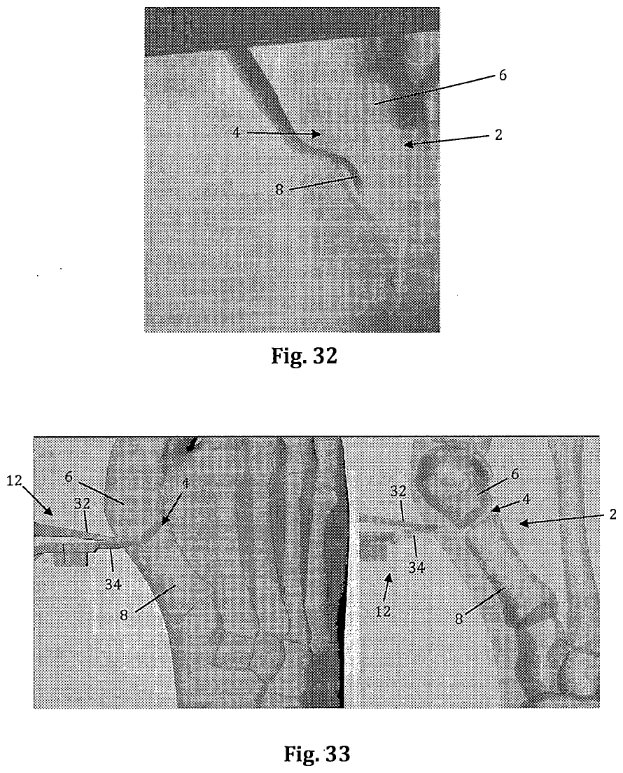

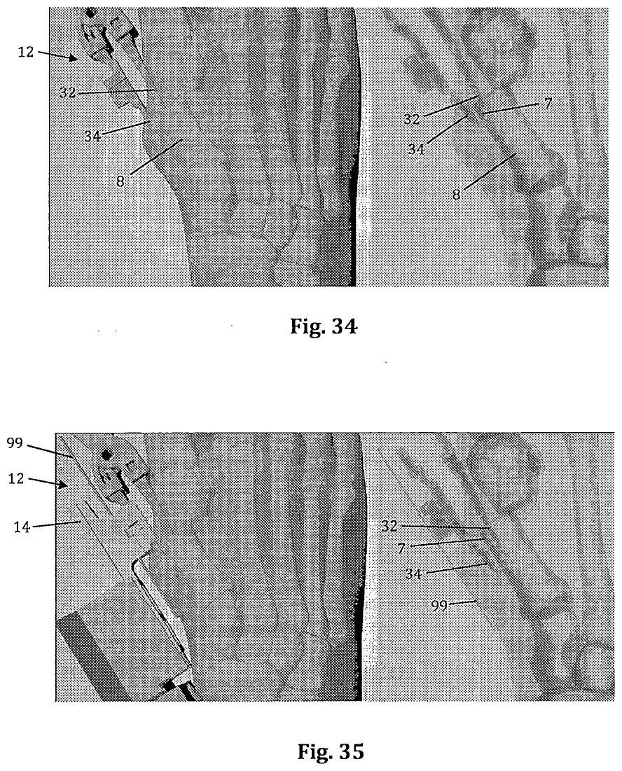

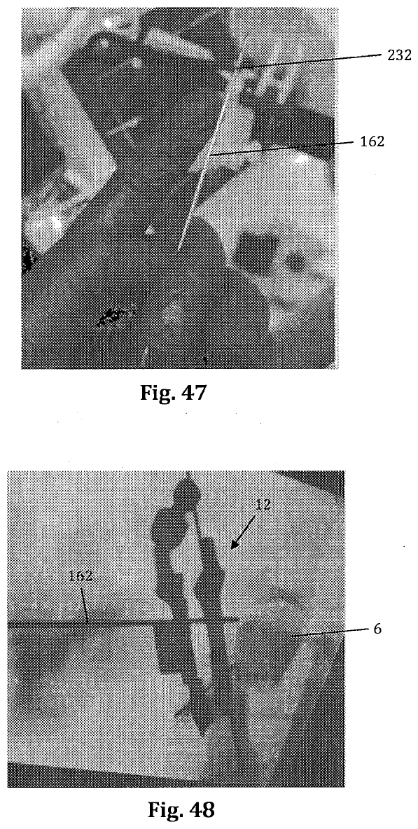

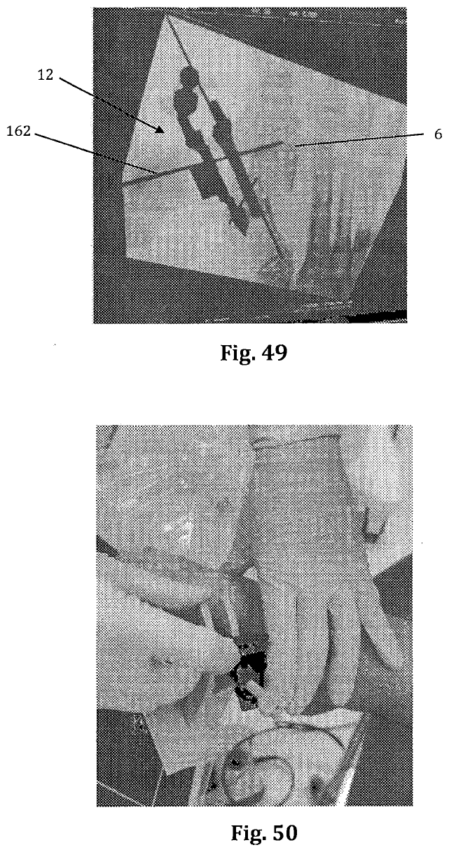

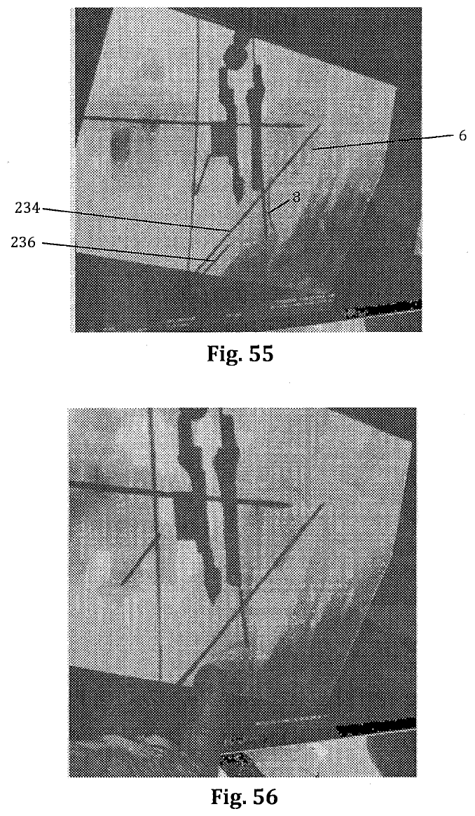

FIG. 31 is a flowchart illustrating various steps in a method 250 of using and assembling the intramedullary targeting guide assembly 10 of the present disclosure. FIGS. 32-69 illustrate the various steps in a method 250 of using and assembling the intramedullary targeting guide assembly 10 of presented in the flowchart of FIG. 31. The targeting guide assembly 10 may be used in a wide range of orthopedic procedures, however for the purpose of illustration the steps presented herein are directed to a specific example of bunion surgery such as the method shown and described in commonly owned U.S. Pat. No. 12,251,116 (“the '116 patent”) the entire contents of which are hereby incorporated by reference into this disclosure as if set forth fully herein.

By way of example, FIG. 32 illustrates a first metatarsal bone 2 of a patient's foot in which an instrument has been inserted through an osteotomy 4 between the metatarsal head 6 (or “distal fragment 6”) and metatarsal shaft 8. In some embodiments, the instrument may be inserted and moved or wiggled to loosen the fracture for repair. Accordingly, a first step 252 in the method 250 of using and assembling the intramedullary targeting guide assembly 10 is to perform an osteotomy in a target bone segment (e.g., metatarsal shaft 8) to create an opening into the intramedullary canal of the target bone segment.

In some embodiments, once the osteotomy 4 is prepared, a next step 254 in the method 250 of using and assembling the intramedullary targeting guide assembly 10 is to introduce the clamp 12 to the surgical site, for example by inserting the intramedullary tip jaw 32 directly into the intramedullary canal of the metatarsal shaft 8, as shown by way of example in FIGS. 33-35. In some embodiments, the intramedullary tip jaw 32 is inserted into the intramedullary canal that has been exposed by way of the osteotomy 4, and advanced longitudinally in a proximal direction (relative to the bone) into the canal without penetrating the cortical wall of the metatarsal shaft. In some embodiments, this longitudinal advancement may be a distance between 3 mm and 5 mm. In some embodiments, the shovel tip jaw 34 is simultaneously inserted into the surgical target site subcutaneously but on the outside of the metatarsal shaft 8 such that a portion of cortical wall 7 is between the intramedullary tip jaw 32 and the shovel tip jaw 34.

In some embodiments, once the intramedullary tip jaw 32 and the shovel tip jaw 34 have been inserted, a next step 256 of the method of using the intramedullary targeting guide assembly 10 is to couple the rail member 14 to the shovel tip jaw 34 of the clamp 12, as shown by way of example in FIG. 35. In some embodiments, this may be accomplished by advancing the rail member 14 such that the coupling flange 76 of the shovel tip jaw 34 is advanced into the transverse recess 94 of the rail member 14 until the deflectable flange 96 is engaged to secure the coupling, as described above. In some embodiments, the drill guide assembly 16 may be preassembled with the rail member 14 prior to coupling the rail member 14 to the clamp 12.

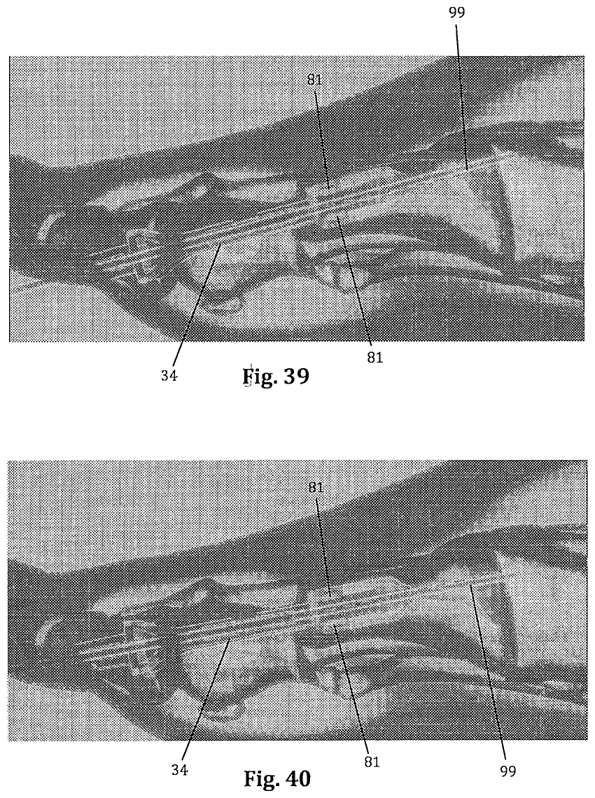

In some embodiments, a next step 258 in the method 250 of using and assembling the intramedullary targeting guide assembly 10 is to move the clamp 12 into position on plane in the middle of the metatarsal, as shown by way of example in FIGS. 36-40. By way of example, this can be done by manually adjusting the position of the clamp 12 such that the radiographic markers 81 are visible and indicate proper positioning on plane in the middle of the metatarsal bone, as shown by way of example in FIG. 36 and FIG. 38 (e.g., showing lateral view under fluoroscopy). In the example shown, the radiographic markers 81 comprise a pair of holes or voids in the shovel tip jaw 34 having a recognizable shape (e.g., oval, ellipse, rectangular, etc.), and proper rotational alignment is indicated by clear visualization of both shaped holes. By way of example, FIG. 37 illustrates an improper rotational alignment, which is evident in that the radiographic markers 81 are not both clearly visible. Additionally, the external guide wire 99 may also be used to confirm proper positioning of the clamp 12 on plane in the middle of the metatarsal canal, which is represented as a dashed line in FIGS. 38-40. As illustrated in FIG. 38, the external guide wire 99 acts as a third radiographic marker to indicate directional alignment relative to the middle of the canal. By way of example, FIGS. 39-40 illustrate improper directional alignment of the clamp 12. By using three radiographic markers, the intramedullary targeting guide assembly 10 is able to ensure proper positioning of the clamp 12 through triangulation. In some embodiments, one or more radiographic markers, for example radiographic markers 81 positioned on the shovel tip jaw 34, may be positioned subcutaneously or otherwise within the patient's body. In some embodiments, one or more of the radiographic markers, for example the guide wire 99, may be positioned in an exterior aspect of the patient.

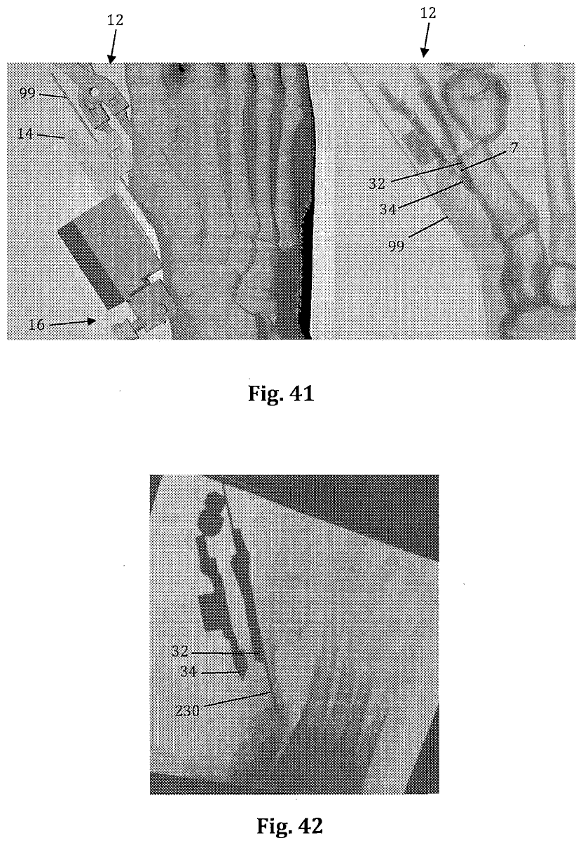

In some embodiments, once proper positioning has been confirmed, a next step 260 in the method 250 of using and assembling the intramedullary targeting guide assembly 10 is to actuate the ring handle 22 to apply a clamping force on the metatarsal shaft 8 such that the cortical wall 7 is captured and held between the intramedullary tip jaw 32 and the shovel tip jaw 34, as shown by way of example in FIG. 41. In some embodiments, the locking element 24 maintains the relative positioning between the intramedullary tip jaw 32 and the shovel tip jaw 34. Once clamped and secured, the targeting guide assembly 10 has set the plane for the bunion fixation surgery and the targeting guide assembly 10 is fixed in the plane of choice without the use of external fixation elements (e.g., K-wires, screws, pins, etc.). By way of example, the targeting guide assembly 10 of the present disclosure is now anchored to the cortex without introducing additional holes into said cortex (e.g., other than the osteotomy).

In some embodiments, once the clamp 12 has been properly positioned, the user may optionally insert a security wire 230 along the intramedullary tip jaw 32 and into the cortical wall of the base of the metatarsal bone, as shown by way of example in FIG. 42. In some embodiments, the security wire 230 may help stabilize the engagement of the clamp 12 to the metatarsal bone 2 and prevent the guide assembly 10 from toggling back and forth, however, this is an optional step as the clamping of the clamp 12 onto the cortical wall 7 will securely hold the targeting guide assembly 10 in plane. In some embodiments, this step may optionally be performed before clamping. In some embodiments, another function of the security wire 230 is to provide a continuing indication of the middle of the metatarsal canal in a top-down (or anterior posterior or A/P) fluoroscopy view, as shown for example in FIG. 42.

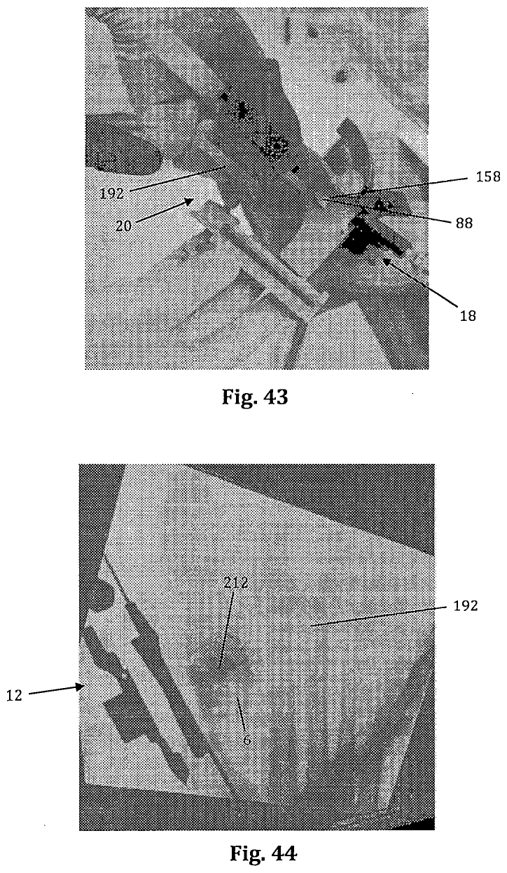

In some embodiments, a next step 262 in the method 250 of using and assembling the intramedullary targeting guide assembly 10 is to couple the rotational assembly 18 to the rail member 14, as shown by way of example in FIG. 43. In some embodiments, this may be accomplished by sliding the coupling aperture 158 of the rotation guide 146 onto the elongated coupling element 88 of the rail member 14 as described above. In some embodiments, the vertical shift assembly 20 may be preassembled with the rotation assembly 18, and the vertical support paddle 192 is positioned under the patient's foot.

In some embodiments, a next step 264 in the method 250 of using and assembling the intramedullary targeting guide assembly 10 is to adjust the medial-lateral position of the vertical support paddle 192 such that the through-hole 212 (e.g., which shows as a distinct dot under fluoroscopy) is positioned at the center of the metatarsal head 6 of the bone under repair, as shown by way of example in FIG. 44. This may be accomplished by manually operating the coupling element 198 of the vertical shift assembly 20 and positioning the vertical support paddle 192 accordingly.

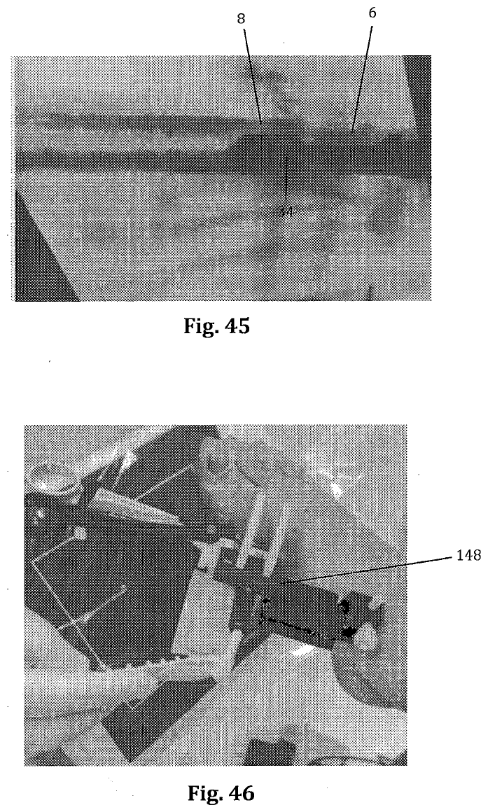

In some embodiments, a next step 266 in the method 250 of using and assembling the intramedullary targeting guide assembly 10 is to adjust the vertical position of the vertical support paddle 192 so that the bone fragments (e.g., metatarsal head 6 and metatarsal shaft 8) are in alignment, as shown by way of example in FIG. 45. By way of example, the vertical support paddle 192 may be used to lift the metatarsal head 6. In some embodiments, this may be accomplished by rotating the vertical shift screw 194 clockwise or counterclockwise to raise or lower the vertical support paddle 192.

In some embodiments, a next step 268 in the method 250 of using and assembling the intramedullary targeting guide assembly 10 is to insert a stabilization wire 162 through the rotation mount 148 and into the middle of the metatarsal head 6 (or “distal fragment”), as shown by way of example in FIGS. 46-49. In some embodiments, the stabilization wire 162 has a laser mark 232 or equivalent that indicates a depth past which the wire should not be advanced. By way of example, the stabilization wire 162 is preferably oriented perpendicular to the clamp 12.

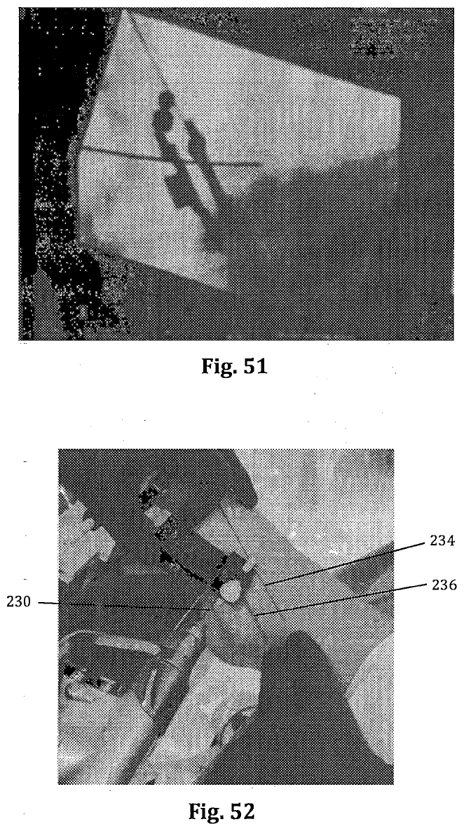

In some embodiments, a next step 270 in the method 250 of using and assembling the intramedullary targeting guide assembly 10 is to rotate the sesamoids into position by using the rotation guide 18, as described above and shown by way of example in FIGS. 50-51.

In some embodiments, a next step 272 in the method 250 of using and assembling the intramedullary targeting guide assembly 10 is to stabilize the distal portion of the intramedullary targeting guide assembly 10 by inserting a stabilizing element such as a security wire 230 through the horizontal slot 116 of the drill guide base 100 and into a bone segment that is not the bone under repair to secure the distal portion of the intramedullary targeting guide assembly 10 in place, as shown by way of example in FIG. 52. For example, in a bunion repair procedure, the security wire 230 may be inserted perpendicularly into the navicular bone to stabilize the distal portion of the targeting guide 10. It should be noted that this step is optional, as the clamp 12 will securely hold the targeting guide assembly 10 in place for the duration of the procedure.

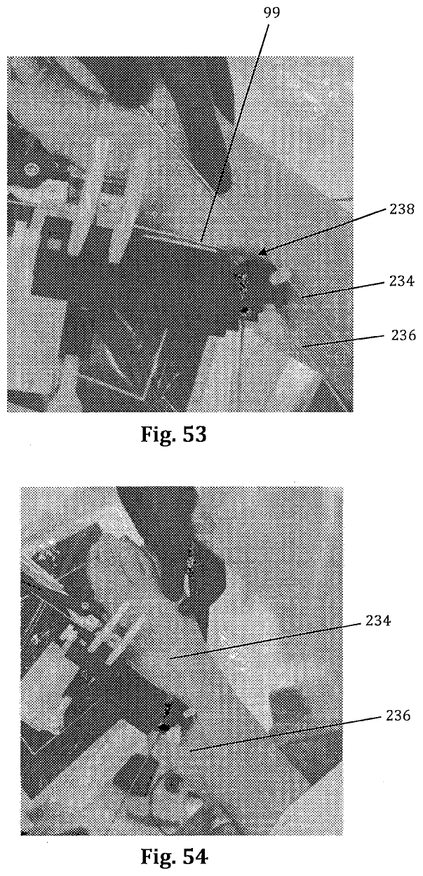

In some embodiments, a next step 274 in the method 250 of using and assembling the intramedullary targeting guide assembly 10 is to determine the location of the proximal incision, as shown by way of example in FIG. 53. To accomplish this, in some embodiments a blunt-tip external guide wire 99 may be inserted through the elongated guide aperture 97 of the rail member 14 in a distal direction relative to the rail member 14 (which is in a proximal direction relative to the patient). This external guide wire 99 may be inserted such that the tip reaches skin but does not penetrate skin. Meanwhile, the drill guide assembly 16 may be provided with a template wire 234 and insertion wire 236 preloaded into the guide aperture 134 and central guide aperture 136, respectively, of the drill guide pivot 128. As noted above, the template wire 234 extends over the patient skin and helps the surgeon determine the appropriate trajectory of anchor insertion, while the insertion wire 236 is inserted into the patient and guides the fixation screw to its proper location. Initially, however, the insertion wire 236 may be advanced through the central guide aperture 136 until the tip reaches skin but does not penetrate skin. When the proper trajectory is determined, the tip of the insertion wire 236 should meet the tip of the external guide wire 99, and the location of this meeting is the proximal incision location 238.

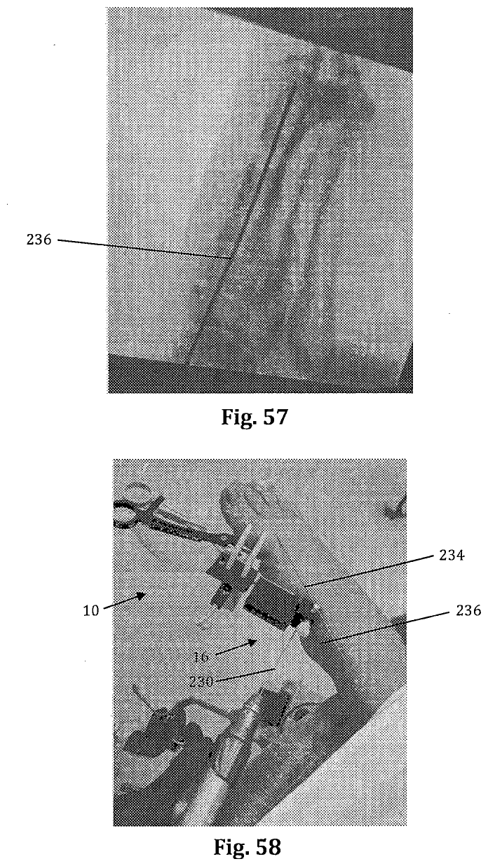

In some embodiments, a next step 276 in the method 250 of using and assembling the intramedullary targeting guide assembly 10 is to determine the insertion trajectory. As shown by way of example in FIGS. 54-56, this may be accomplished by positioning the template wire 234 above the skin so that when viewing the target area under fluoroscopy, the template wire 234 appears to extend through the metatarsal shaft 8 and into the lateral third of the metatarsal head 6. The patient's foot may be rotated so that the insertion wire 236 and template wire 234 appear as one single wire. When this happens, the surgeon may advance the insertion wire 236 along this trajectory so that the tip of the insertion wire 236 is in the lateral third of the metatarsal head 6 and the insertion wire 236 extends through the middle of the metatarsal when viewed laterally (e.g., FIG. 57).

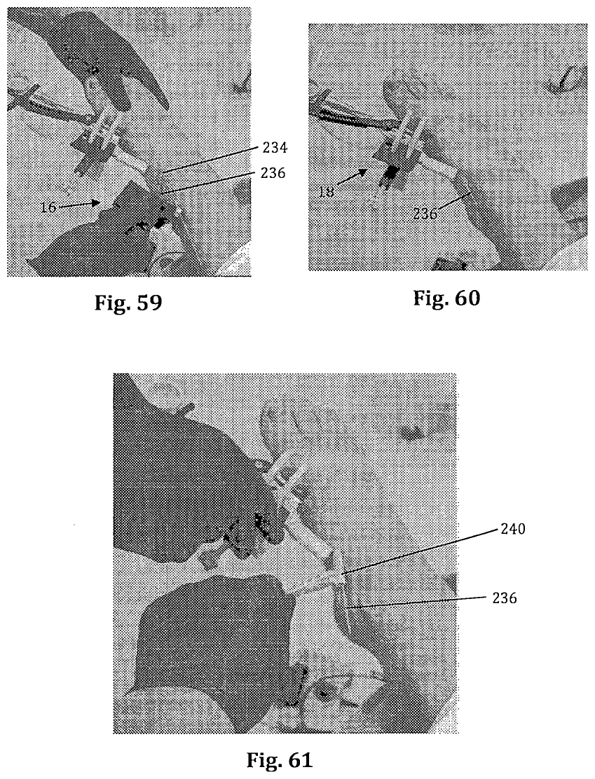

In some embodiments, once the insertion wire 236 has been placed in the proper position in the metatarsal head 6 (e.g., lateral third) and in the proper trajectory through the metatarsal shaft 8, it is time to drill the bone and place the implant. Before this can be done, however, at next step 278 in the method 250 of using and assembling the intramedullary targeting guide assembly 10 is to remove the various positioning wires (e.g., including the template wire 234, external guide wire 99, security wire 230, etc.) along with the drill guide assembly 16. By way of example, the external guide wire 99 may be removed first (and it may be removed immediately following determining the location of the proximal incision), followed by the security wire 230, as shown by way of example in FIG. 58. Once the security wire 230 has been removed, the drill guide assembly 16 may be removed by squeezing the handle member 126 of the coupling element 104 and sliding proximally along and ultimately off the insertion wire 236, as shown by way of example in FIG. 59. The remaining portion of the intramedullary targeting guide assembly 10 at this point include the clamp 12, rail member 14, rotation assembly 16, and vertical shift assembly 18 (as shown in FIG. 60), which are all operating to hold the patient's foot in the proper orientation.

As shown by way of example in FIG. 61, a next step 280 in the method 250 of using and assembling the intramedullary targeting guide assembly 10 is to advance a drill guide 240 over the insertion wire 236 to the proximal incision. If necessary, the proximal incision may be enlarged. In some embodiments, a next step 282 in the method 250 of using and assembling the intramedullary targeting guide assembly 10 is hole drilling and screw placement in the target bone segments.

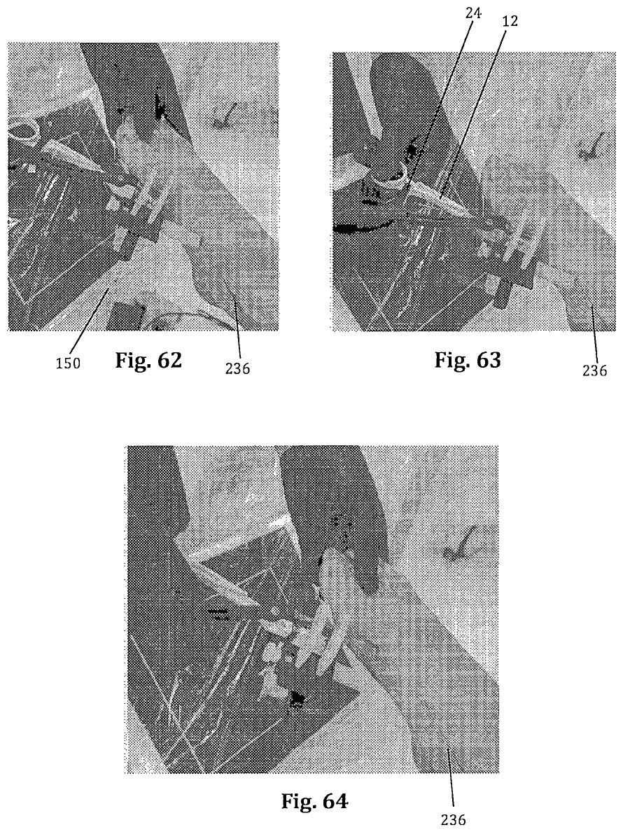

Referring to FIGS. 62-64, after placement of the anchor(s), a next step 284 in the method 250 of using and assembling the intramedullary targeting guide assembly 10 is to remove the remainder of the intramedullary targeting guide assembly 10 from the target site. To accomplish this, a first step is to unscrew the horizontal shift screw 150 and remove the stabilization wire 162 from the metatarsal head 6. Next, the locking element (e.g., ratchet mechanism) 24 may be released to loosen the clamp 12, following which the security wire 230 that was inserted along the intramedullary tip jaw 32 may then be removed. Once the security wire 230 has been removed, the clamp 12 (e.g., with rail member 14, rotation assembly 18, and vertical shift assembly 20 still attached) can be pulled out of the intramedullary canal and out of the incision. The distal incision may be closed at this point. Next, the insertion wire 236 may be removed and the proximal incision closed.

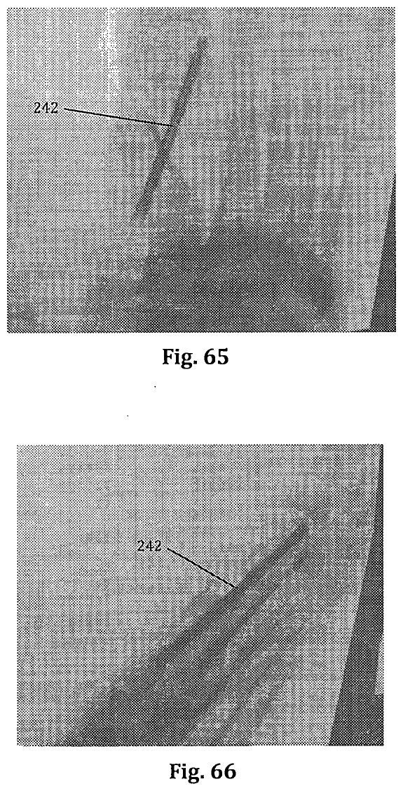

By way of example, FIGS. 65-66 illustrate top and side views, respectively, of an example of the final screw 242 placement after using the intramedullary targeting guide assembly 10 of the present disclosure in a bunion repair surgery.

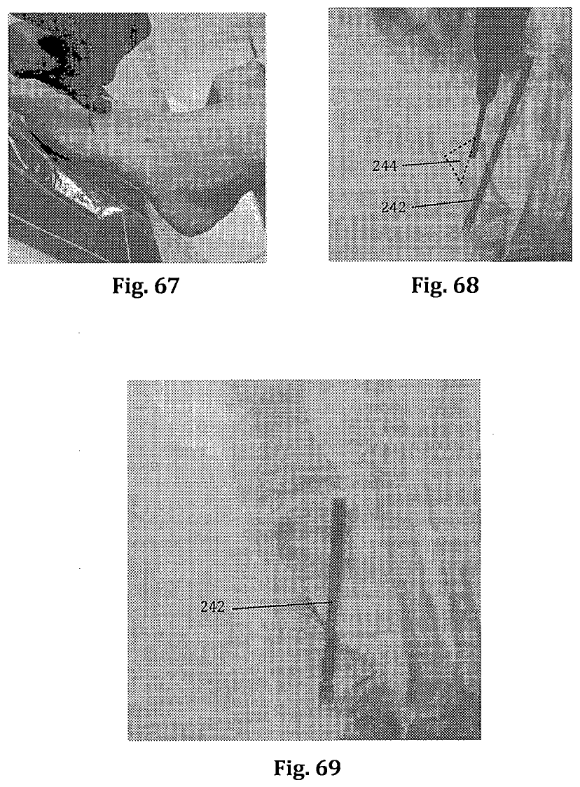

By way of example, FIGS. 67-69 illustrate removal of the triangular piece of bone 244 referred to as a prominent redundant bone using the technique shown and described in the '746 application.

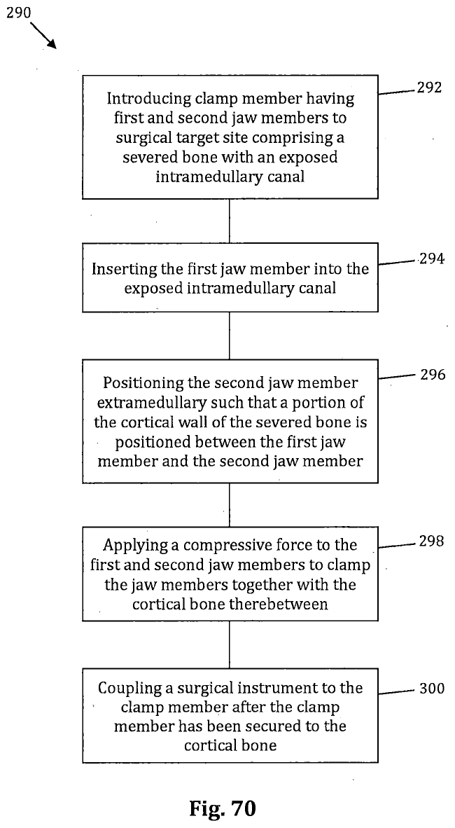

In some embodiments, in addition to the method 250 of using the intramedullary targeting guide assembly 10 in the course of a minimally invasive bunion surgery described above, this disclosure describes several other novel methods. For example, FIG. 70 is a flowchart describing an example method 290 of registering a surgical instrument (e.g., targeting guide 10 or other surgical instrument) to a severed bone segment of a patient by clamping to a cortical wall without penetrating through the cortical wall. In some embodiments, this method is performed in the context of an osteotomy procedure or in a similar fracture scenario which produces the severed bone segment.

In some embodiments, a first step 292 in the method 290 of registering a surgical instrument to a severed bone of a patient includes introducing a clamp member to a surgical target site comprising the severed bone, the clamp member comprising a first jaw member hingedly attached to a second jaw member.

In some embodiments, a next step 294 in the method 290 of registering a surgical instrument to a severed bone of a patient is inserting the first jaw member of the clamp member into an exposed intramedullary canal of the severed bone without penetrating the cortical wall of the severed bone.

In some embodiments, a next step 296 in the method 290 of registering a surgical instrument to a severed bone of a patient is positioning the second jaw member extramedullary such that a portion of the cortical wall of the severed bone is positioned between the first jaw member and the second jaw member. In some embodiments, the second jaw member is introduced subcutaneously.

In some embodiments, a next step 298 in the method 290 of registering a surgical instrument to a severed bone of a patient is applying a compressive force to the first and second jaw members to urge the first and second jaw members toward one another with the cortical bone positioned therebetween, thereby securing the clamp member to the cortical bone without creating additional holes in the cortical bone.

In some embodiments, a next step 300 in the method 290 of registering a surgical instrument to a severed bone of a patient is attaching a surgical instrument to the secured clamp member. In some embodiments, the surgical instrument is a targeting guide, however it is contemplated that other surgical instruments may be registered to bone using this technique.

In some embodiments, this disclosure describes a method of aligning a surgical instrument placed at least partially within bone using a plurality of radiographic markers to triangulate a position of the surgical instrument under fluoroscopy in at least two planes. In some embodiments, first and second radiographic markers of the plurality of radiographic markers are used to verify positioning of the surgical instrument in a first plane. In some embodiments, a third radiographic marker of the plurality of radiographic markers is used to verify positioning of the surgical instrument in a second plane, the second plane being different from the first plane. In some embodiments, the first and second radiographic markers are positioned within a patient's body. In some embodiments, the first and second radiographic markers are configured to verify rotational alignment. In some embodiments, the third radiographic marker is positioned exterior to the patient's body. In some embodiments, the third radiographic marker is configured to verify directional alignment.

The term “coupled” is defined as connected, although not necessarily directly, and not necessarily mechanically. The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way but may also be configured in ways that are not listed.

All patents and publications mentioned in this specification are indicative of the levels of those skilled in the art to which the disclosure pertains. It is to be understood that while a certain form of the disclosure is illustrated, it is not to be limited to the specific form or arrangement herein described and shown. It will be apparent to those skilled in the art that various changes may be made without departing from the scope of the disclosure and the disclosure is not to be considered limited to what is shown and described in the specification and any drawings/figures included herein.

One skilled in the art will readily appreciate that the present disclosure is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the disclosure and are defined by the scope of the appended claims. Although the disclosure has been described in connection with specific preferred embodiments, it should be understood that the disclosure as claimed should not be unduly limited to such specific embodiments. Indeed, various modifications of the described modes for carrying out the disclosure which are obvious to those skilled in the art are intended to be within the scope of the following claims.

What is claimed is:

1. A surgical targeting guide system configured for in situ assembly, comprising:
a clamp member defining a proximal end of the surgical targeting guide system, the clamp member having a first elongated jaw member hingedly attached to a second elongated jaw member and a locking element configured to secure the first and second elongated jaw members in a clamped position, the clamp member further comprising a longitudinal axis and a transverse axis defining an alignment plane extending through the first and second elongated jaw members;
a rail member configured for in situ coupling with the clamp such that the rail member is coplanar with the alignment plane;
a drill guide assembly configured for in situ coupling with the rail member, the drill guide assembly defining a distal end of the surgical targeting guide assembly and comprising a coupling element, a housing, and a pivot member, the coupling element configured to removably couple the drill guide assembly to the rail member, the pivot member positioned within the housing and configured to pivot about a vertical axis perpendicular to the longitudinal axis, the pivot member further comprising a central guide aperture configured to receive a guide wire therethrough;
a rotation guide assembly configured for in situ slidable coupling with the rail member, the rotation guide assembly including a base member having at least one curved flange and a rotation mount slidably coupled with the at least one curved flange; and
a vertical shift assembly configured for in situ coupling with the rotation guide assembly, the vertical shift assembly including a base member including a coupling element configured to removably couple the vertical shift assembly to the rotation guide assembly, a support paddle, and an actuator configured to effect displacement of the support paddle in a direction perpendicular to the alignment plane.

2. The system of claim 1, wherein the locking element of the clamp member comprises a ratchet mechanism or a spin lock mechanism.

3. The system of claim 1, wherein the first jaw member has a tapered tip configured to facilitate insertion into an intramedullary canal of a bone.

4. The system of claim 1, wherein the second jaw member has a shovel tip including a bone engagement portion configured to interact with extramedullary bone to prevent movement of the shovel tip relative to the extramedullary bone.

5. The system of claim 1, wherein the rail member comprises an elongated coupling element configured to slidably engage the rotation guide assembly.

6. The system of claim 1, wherein the rail member is coupled with the clamp by way of an interaction with an engagement feature on the second jaw member.

7. The system of claim 1, wherein the drill guide assembly comprises a coupling element configured to removably couple the drill guide assembly to the rail member.

8. The system of claim 1, wherein the drill guide assembly further comprises a locking element actuatable to lock the pivot member in a targeted position.

9. The system of claim 1, wherein the rotation assembly includes a transverse actuator configured to couple with a bone engagement element such that the bone engagement element is controllably adjustable upon actuation of the transverse actuator.

10. A method of aligning a surgical instrument, comprising:
using a plurality of radiographic markers associated with the surgical instrument to triangulate a position under fluoroscopy in at least two planes;
wherein first and second radiographic markers of the plurality of radiographic markers are used to verify positioning of the surgical instrument in a first plane; and
wherein a third radiographic marker of the plurality of radiographic markers is used to verify positioning of the surgical instrument in a second plane, the second plane being different from the first plane.

11. The method of claim 10, wherein the first and second radiographic markers are positioned within a patient's body.

12. The method of claim 10, wherein the first and second radiographic markers are configured to verify rotational alignment.

13. The method of claim 10, wherein the third radiographic marker is positioned exterior to the patient's body.

14. The method of claim 10, wherein the third radiographic marker is configured to verify directional alignment.

15. A method of registering a surgical instrument to a severed bone segment of a patient, comprising:
introducing a clamp member to a surgical target site comprising the severed bone, the clamp member comprising a first jaw member hingedly coupled to a second jaw member;
inserting the first jaw member into an exposed intramedullary canal of the severed bone without penetrating the cortical wall of the severed bone;
positioning the second jaw member extramedullary such that a portion of the cortical wall of the severed bone is positioned between the first jaw member and the second jaw member;
applying a compressive force to the first and second jaw members to urge the first and second jaw members toward one another with the cortical bone positioned therebetween, securing the clamp member to the cortical bone without creating additional holes in the cortical bone; and
coupling a surgical instrument to the clamp member.

16. The method of claim 15, wherein the surgical instrument is coupled to the clamp member after the clamp member has been secured to the cortical bone.

17. The method of claim 15, wherein the surgical instrument is a targeting guide.

18. The method of claim 15, wherein the step of coupling a surgical instrument to the clamp member occurs before the step of applying a compressive force to the first and second jaw members.

19. The method of claim 15, wherein the severed bone segment is a result of at least one of an osteotomy procedure and a fracture event.

20. The method of claim 15, further comprising the step of:
adjusting the position of the clamp member after completing the steps of inserting the first jaw member and positioning the second jaw member.
